(19) United States
(12) Patent Application Publication
(10) Pub. No.: US 2025/0362283 A1
(43) Pub. Date: Nov. 27, 2025

(54) DETECTING SAG VALUE OF WEIGHTING MATERIAL IN DRILLING FLUID IN BOREHOLE

(71) Applicant: KING FAHD UNIVERSITY OF PETROLEUM AND MINERALS, Dhahran (SA)

(72) Inventors: Panagiotis KIRMIZAKIS, Dhahran (SA); Ali Abdulwahad OSHAISH, Dhahran (SA); Ammar EL-HUSSEINY, Dhahran (SA); Panteleimon SOUPIOS, Dhahran (SA); Mohamed MAHMOUD, Dhahran (SA)

(73) Assignee: KING FAHD UNIVERSITY OF PETROLEUM AND MINERALS, Dhahran (SA)

(21) Appl. No.: 18/679,719

(22) Filed: May 31, 2024

(30) Foreign Application Priority Data
May 26, 2024 (SA) 1020242868

(51) Int. Cl. G01N 33/28; E21B 21/06; G01N 9/00; G01N 15/06

(52) U.S. Cl. CPC G01N 33/2823; E21B 21/06; G01N 9/00; G01N 15/0656

(57) ABSTRACT

A method of detecting a sag value of a weighting material in a drilling fluid in a borehole by injecting an alternating current into the drilling fluid in a frequency range of 0.1-10,000 hertz (Hz) over a time period of 1-6 hours. The method includes measuring real-time data of a real conductivity and an impedance of the drilling fluid based by spectral induced polarization (SIP) of the drilling fluid in the borehole and/or during the injecting. Calculating a first density and a second density of the drilling fluid at a first elevation and a second elevation, respectively, each based on a variation in the real conductivity and the impedance, and calculating the sag value of the weighting material in the drilling fluid based on the first and second densities.

### CROSS REFERENCE TO RELATED APPLICATION

The present disclosure claims the benefit of Saudi Patent Application No. 1020242868 filed on May 26, 2024, with the Saudi Authority for Intellectual Property Office, which is incorporated herein by reference in its entirety.

### BACKGROUND

### Technical Field

The present disclosure relates to the field of drilling fluid management within the oil and gas industry, and more specifically to a method for early detection and monitoring of sagging in weighting materials, such as barite and calcite, used in drilling fluids within boreholes.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

In oil and gas extraction, the drilling fluids, or muds, are used for the successful drilling of boreholes into the earth. These fluids are complex mixtures designed to facilitate the drilling process by carrying drill cuttings to the surface, cooling and lubricating the drill bit, and maintaining hydrostatic pressure to prevent well blowouts. Weighting materials, which are solids with high specific gravity, are added to drilling fluids to enhance their density and control pressure during drilling operations. Barite (Barium sulfate, BaSO₄) and Calcium carbonate (CaCO₃) are commonly used for this purpose [See: Mohamed, A., Basfar, S., Elkatatny, S., & Al-Majed, A. (2019)—Prevention of Barite Sag in oil-based drilling fluids using a mixture of barite and ilmenite as weighting material. Sustainability (Switzerland), 11(20), 5617; Mohamed, A., Al-Afnan, S., Elkatatny, S., & Hussein, L (2020)—Prevention of barite sag in water-based drilling fluids by a urea-based additive for drilling deep formations. Sustainability (Switzerland), 12(7), 2719; Nguyen, T., Silva, C., Dare, A., Saasen, A., & Al-, E. (2016)—Optimization of Calcium Carbonate Particles and pH in Oil Based Drilling Fluids to Minimize Barite Sag; Ofei, T. N., Lund, B., & Saasen, A. (2021)—Effect of particle number density on rheological properties and barite sag in oil-based drilling fluids. Journal of Petroleum Science and Engineering, 206 (January)]. Solid separation can occur during drilling, affecting the consistency of the drilling fluid column and leading to uneven hydrostatic pressure [See: Amani, M. (2019)—An Investigation of the Factors Affecting Barite Sagging Related to Drilling Fluid Properties]. This phenomenon, known as weighting material sag, can occur both when the drilling fluid is stationary (such as in tanks or during operational pauses) and when it is flowing in the annulus [See: Mohamed et al., 2020; Mohamed, A., Basfar, S., Elkatatny, S., & Al-Majed, A. (2021)—Enhancement of Static and Dynamic Sag Performance of Water-Based Mud Using a Synthetic Clay. ACS Omega, 6(12), 8179-8188; Murphy, R., Jamison, D., Hemphill, T., Bell, S., & Albrecht, C. (2008)—Measuring and predicting dynamic sag. SPE Drilling and Completion, 23(2), 142-149].

Weighting material sag adversely impacts drilling operations and well control by altering pressure containment capabilities of the drilling fluid. In severe cases, solids can settle at bottom of the well, obstructing the drill bit or causing the pipe to get stuck [See: Amani, 2019; Amani et al., 2018; Jamison, D. E., & Murphy Jr, R. J. (2003)—Apparatus and method for analyzing well fluid sag (Issue 19); Miller, J. J., Braley, N., Siems, D. R., Jamison, D. E., & Baker, P. (2013)—Drilling Fluids Having Reduced SAG Potential and Related Methods; Mohamed et al., 2019; Odaba i, A. (2015)—An Experimental Study of Particle Size and Concentration Effects of Calcium Carbonate on Rheological and Filtration Properties of Drill-in Fluids (Issue May). Middle East Technical University; Omland, T. H. (2009)—Particle Settling in non-Newtonian Drilling Fluids; Wagle, V., Maghrabi, S., & Kulkarni, D. (2013)—Formulating sag-resistant, low-gravity solids-free invert emulsion fluids. SPE Middle East Oil and Gas Show and Conference, MEOS, Proceedings, 1, 508-519]. Solids separation is an inevitable key aspect associated with this process, where fluids with fewer solids fail to maintain the necessary fluid density and hydrostatic pressure for well control. Factors like high pressure, temperature, and the well's inclination angle significantly influence drilling fluid design and sag behavior. Temperature has been identified as a critical factor, with sag tendency increasing proportionally with temperature. The angle of inclination also plays a crucial role, with a 45-degree angle often leading to the highest sag factor [See: Amani, 2019; Amani, M., Roustazadeh, A., Farooq Zia, M., Al-Emadi, S., Carvero, A., & Yrac, R. (2018)—An Experimental Analysis of the Factors Affecting Barite Segregation in Water Based Drilling Fluids. International Journal of Petroleum and Petrochemical Engineering (IJPPE), 4 (December 2018), 14-31; Omland, 2009; Tor H. Omland, Arild Saasen, & Per Amund Amundsen. (2007)—Detection Techniques Determining Weighting Material Sag in Drilling Fluid and Relationship to Rheology. Annual Transactions of The Nordic Rheology Society, 15; Wagle et al., 2013]. Research by Ofei et al. indicates that the number of particles in the drilling fluid can also impact sag, with more particles contributing to better sag prevention.

Since the 1980s, various techniques have been developed to assess weighting material sag. Initial methods primarily examined the rheological attributes of drilling fluids, exploring how these properties related to sag in pipes positioned at an angle [See: Omland, T. H., Saasen, A., Zwaag, C. v.d., & Amundsen, P. A. (2007)—The effect of weighting material sag on drilling operation efficiency. SPE—Asia Pacific Oil and Gas Conference, 2, 1013-1017]. Another prominent technique involves analyzing variations in the density of drilling fluids. This method has become a standard for sag testing, particularly under static conditions. This density-based test measures density of the drilling fluid at the top and bottom of a column after it has been stored at high temperatures without movement. The sag factor is then calculated using a specific formula (eq. 1), with a factor below 0.5 indicating that the drilling fluid is unlikely to experience sag (‘sag-safe’), while a factor of 0.53 or higher signals a high likelihood of sagging.

$$SAG Factor = \frac{{density}_{top}}{{density}_{b o t t o m} + {density}_{top}} \qquad ( 1 )$$

In the effort to better understand and manage sagging in drilling operations, researchers consider numerous factors. These include the rate of penetration, the efficiency of mud pumps, and the speed of pipe rotation [See: Calgada, L. A., Duque Neto, O. A., Magalhdes, S. C., Scheid, C. M., Borges Filho, M. N., & Waldmann, A. T. A. (2015)—Evaluation of suspension flow and particulate materials for control of fluid losses in drilling operation. Journal of Petroleum Science and Engineering, 131, 1-10]. Based on these and other elements, various tests have been developed. These include ultrasonic weight measurement, flow loops, viscometers, high-angle sag tests, among others [See: Bern, P. A., Zamora, M., Hemphill, A. T., Marshall, D., Omland, T. H., & Morton, E. K. (2010)—Field Monitoring of Weight-Material Sag. The 2010 AADE Fluids Conference and Exhibition; Dehghani, F., Kalantariasl, A., Saboori, R., Sabbaghi, S., & Peyvandi, K. (2019)—Performance of carbonate calcium nanoparticles as filtration loss control agent of water-based drilling fluid. SN Applied Sciences, 1(11), 1-8; Fort, J. A., Bamberger, J. A., Bates, J. M., Enderlin, C. W., & Elmore, M. R. (1993)— 1/12-scale physical modeling experiments in support of tank 241-SY-101 hydrogen mitigation; Murphy et al., 2008; Omland, 2009; Shen, C., & Lemmin, U. (1996)—Ultrasonic measurements of suspended sediments: A concentration profiling system with attenuation compensation. Measurement Science and Technology, 7(9), 1191-1194; Wagle et al., 2013]. However, these methods have their limitations. They often fall short in terms of time and cost efficiency, and may require complex, bulky equipment that may be impractical for certain locations. Another significant challenge is accuracy. Some techniques, like density measurement using weight-over-volume methods, suffer from issues like lack of reproducibility and precision, often compounded by human error.

US 20140172305A1 describes systems and methods for the real-time detection and measurement of sag within a deviated borehole. This references provides a method which includes measuring a first pressure at a first time at a point within the borehole, predicting a characteristic of the drilling fluid at the point using a computer model, thereby obtaining a predicted characteristic, calculating the characteristic based on the first pressure, thereby obtaining a calculated characteristic, and determining whether sag has occurred based on a comparison between the calculated characteristic and the predicted characteristic [Abstract]. However, this reference does not disclose implementation of the technique of spectral induced polarization (SIP) for measuring real-time data of a real conductivity and an impedance of the drilling fluid, to be used for calculating the sag value of the weighting material in the drilling fluid.

U.S. Pat. No. 4,359,687A describes an apparatus for borehole measurements of the induced polarization of earth formations. The apparatus consists of an induced polarization logger capable of measuring both in-phase and quadrature conductivities in the frequency domain. A method is described which uses these measurements to determine cation exchange capacity per unit pore volume, Qᵥ, brine conductivity, C_{w}, and oil and water saturations, S.ₒ and S_{w}, in shaly sands. However, this reference does not disclose implementation of the technique of spectral induced polarization (SIP) for measuring real-time data of a real conductivity and an impedance of the drilling fluid, to be used for calculating the sag value of the weighting material in the drilling fluid.

Each of the aforementioned references suffers from one or more drawbacks. Most of these conventional solutions require the extraction of drilling fluid samples from the borehole for analysis, which is not only time-consuming but also interrupts the drilling process. Furthermore, these techniques often fail to provide real-time data on the sagging behavior of the weighting materials, which limits their effectiveness in preventing sag-related issues during drilling operations. Additionally, the laboratory-based analyses, as implemented in some cases, may not accurately replicate the complex conditions of a wellbore, leading to discrepancies between predicted and actual sag behavior.

Accordingly, it is one object of the present disclosure to provide a method and system for detecting and monitoring the sag of weighting materials in drilling fluids directly within the borehole with real-time monitoring capabilities, eliminating the need for fluid extraction and laboratory analysis. The method and system of the present disclosure provides immediate feedback on the condition of the drilling fluid, allowing for timely adjustments to composition of the drilling fluid or drilling parameters to mitigate the risk of sagging. The present disclosure aims to address these requirements, leveraging advanced techniques to monitor the density and stability of drilling fluids in situ, thus enhancing the efficiency and safety of drilling operations.

### SUMMARY

In an aspect, a method of detecting a sag value of a weighting material in a drilling fluid in a borehole is described. The method comprises injecting an alternating current into the drilling fluid in a frequency range of 0.1-10,000 hertz (Hz) over a time period of 1-6 hours. The method further comprises measuring real-time data of a real conductivity and an impedance of the drilling fluid based on a spectral induced polarization (SIP) of the drilling fluid during the injecting. The method further comprises calculating a first density and a second density of the drilling fluid at a first elevation and a second elevation, respectively, each based on a variation in the real conductivity and the impedance. The method further comprises calculating the sag value of the weighting material in the drilling fluid based on the first and second densities. Herein, the variation in the real conductivity and the impedance directly relates to changes in a concentration of particles of the weighting material in the drilling fluid.

In some embodiments, the method does not comprise extracting the drilling fluid from the borehole for the measuring.

In some embodiments, an amplitude of the alternating current is in a range of 1-10 volts (V).

In some embodiments, the frequency range is 1000-10,000 Hz, and wherein the time period is in a range of from 1-3 hours.

In some embodiments, the method further comprises measuring a phase shift and an imaginary conductivity of the drilling fluid at different elevations of the borehole.

In some embodiments, the borehole has a depth of less than 1 km and wherein the injecting occurs concurrently in a top, a middle, and a bottom of the borehole.

In some embodiments, the method further comprises calculating the concentration of the particles of the weighting material in the drilling fluid based on the real conductivity and the impedance.

In some embodiments, the drilling fluid comprises about 40-60 wt. % barite, based on a total weight of the drilling fluid.

In some embodiments, the drilling fluid further comprises 40-60 wt. % water, 0.1-1 wt. % of a polymer, and 0.1-1 wt. % of a starch, each based on the total weight of the drilling fluid.

In some embodiments, the first and second densities of the drilling fluid can be determined according to the equation d_{barite}=(Z+575.37)/381.02. Herein, d_{barite} denotes the first and second densities of the drilling fluid comprising barite, respectively; and Z denotes the first and second impedance of the drilling fluid, respectively.

In some embodiments, the drilling fluid comprises 1-10 wt. % calcite, based on a total weight of the drilling fluid.

In some embodiments, the drilling fluid further comprises 1-10 wt. % bentonite and 80-98 wt. % water, each based on a total weight of the drilling fluid.

In some embodiments, the first and second densities of the drilling fluid can be determined according to the equation d_{calcite}=(Z+690.76)/902.16. Herein, d_{calcite} denotes the first and second densities of the drilling fluid comprising calcite, respectively; and Z denotes the first and second impedance of the drilling fluid, respectively.

In some embodiments, the variation in the real conductivity is 1-20 μS/cm compared to an initial real conductivity.

In some embodiments, the variation in the impedance is 1-20 ohms compared to an initial impedance.

In some embodiments, the drilling fluid has a salinity of 1 to 1000 ppt.

In some embodiments, the drilling fluid is in a reservoir and the reservoir has a temperature of 100-300° C.

In some embodiments, the drilling fluid is in a subterranean borehole having a depth of at least 500 m during the injecting and the measuring.

In some embodiments, the drilling fluid is in a horizontal well during the injecting and the measuring.

In some embodiments, the drilling fluid is in a horizontal well during the injecting and the measuring.

In some embodiments, the measuring is downhole in a well during the injecting and the measuring.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an exemplary flowchart of a method of detecting a sag value of a weighting material in a drilling fluid in a borehole, according to certain embodiments.

FIG. 2 is a schematic illustration of a system including a test column for conducting Spectral Induced Polarization (SIP) tests, according to certain embodiments.

FIG. 3A is a graphical representation illustrating exemplary plot(s) of phase shift in SIP measurements over a broad frequency range (0.1-10,000 Hz) for barite drilling fluid, according to certain embodiments.

FIG. 3B is a graphical representation illustrating exemplary plot(s) of real conductivity in SIP measurements over a broad frequency range (0.1-10,000 Hz) for barite drilling fluid, according to certain embodiments.

FIG. 3C is a graphical representation illustrating exemplary plot(s) of imaginary conductivity in SIP measurements over a broad frequency range (0.1-10,000 Hz) for barite drilling fluid, according to certain embodiments.

FIG. 3D is a graphical representation illustrating exemplary plot(s) of impedance in SIP measurements over a broad frequency range (0.1-10,000 Hz) for barite drilling fluid, according to certain embodiments.

FIG. 4A is a graphical representation illustrating exemplary plot(s) of phase shift in SIP measurements over a broad frequency range (0.1-10,000 Hz) for calcite drilling fluid, according to certain embodiments.

FIG. 4B is a graphical representation illustrating exemplary plot(s) of real conductivity in SIP measurements over a broad frequency range (0.1-10,000 Hz) for calcite drilling fluid, according to certain embodiments.

FIG. 4C is a graphical representation illustrating exemplary plot(s) of imaginary conductivity in SIP measurements over a broad frequency range (0.1-10,000 Hz) for calcite drilling fluid, according to certain embodiments.

FIG. 4D is a graphical representation illustrating exemplary plot(s) of impedance in SIP measurements over a broad frequency range (0.1-10,000 Hz) for calcite drilling fluid, according to certain embodiments.

FIG. 5A is a graphical representation illustrating exemplary plot(s) of timelapse of phase shift in SIP signal measurements for barite drilling fluid at 10 kHz, according to certain embodiments.

FIG. 5B is a graphical representation illustrating exemplary plot(s) of timelapse of real conductivity in SIP signal measurements for barite drilling fluid at 10 kHz, according to certain embodiments.

FIG. 5C is a graphical representation illustrating exemplary plot(s) of timelapse of imaginary conductivity in SIP signal measurements for barite drilling fluid at 10 kHz, according to certain embodiments.

FIG. 5D is a graphical representation illustrating exemplary plot(s) of timelapse of impedance in SIP signal measurements for barite drilling fluid at 10 kHz, according to certain embodiments.

FIG. 6A is a graphical representation illustrating exemplary plot(s) of timelapse of phase shift in SIP signal measurements for calcite drilling fluid at 10 kHz, according to certain embodiments.

FIG. 6B is a graphical representation illustrating exemplary plot(s) of timelapse of real conductivity in SIP signal measurements for calcite drilling fluid at 10 kHz, according to certain embodiments.

FIG. 6C is a graphical representation illustrating exemplary plot(s) of timelapse of imaginary conductivity in SIP signal measurements for calcite drilling fluid at 10 kHz, according to certain embodiments.

FIG. 6D is a graphical representation illustrating exemplary plot(s) of timelapse of impedance in SIP signal measurements for calcite drilling fluid at 10 kHz, according to certain embodiments.

FIG. 7A is a graphical representation illustrating exemplary plots of phase shift measurements at 8 time periods for upper channel 1 over a broad frequency range (0.1-10,000 Hz) for barite drilling fluid, according to certain embodiments.

FIG. 7B is a graphical representation illustrating exemplary plots of phase shift measurements at 8 time periods for lower channel 3 over a broad frequency range (0.1-10,000 Hz) for barite drilling fluid, according to certain embodiments.

FIG. 8A is a graphical representation illustrating exemplary plots of real conductivity measurements at 8 time periods for upper channel 1 over a broad frequency range (0.1-10,000 Hz) for barite drilling fluid, according to certain embodiments.

FIG. 8B is a graphical representation illustrating exemplary plots of real conductivity measurements at 8 time periods for lower channel 3 over a broad frequency range (0.1-10,000 Hz) for barite drilling fluid, according to certain embodiments.

FIG. 9A is a graphical representation illustrating exemplary plots of imaginary conductivity measurements at 8 time periods for upper channel 1 over a broad frequency range (0.1-10,000 Hz) for barite drilling fluid, according to certain embodiments.

FIG. 9B is a graphical representation illustrating exemplary plots of imaginary conductivity measurements at 8 time periods for lower channel 3 over a broad frequency range (0.1-10,000 Hz) for barite drilling fluid, according to certain embodiments.

FIG. 10A is a graphical representation illustrating exemplary plots of impedance measurements at 8 time periods for upper channel 1 over a broad frequency range (0.1-10,000 Hz) for barite drilling fluid, according to certain embodiments.

FIG. 10B is a graphical representation illustrating exemplary plots of impedance measurements at 8 time periods for lower channel 3 over a broad frequency range (0.1-10,000 Hz) for barite drilling fluid, according to certain embodiments.

FIG. 11A is a graphical representation illustrating exemplary plots of phase shift measurements at 7 time periods for upper channel 1 over a broad frequency range (0.1-10,000 Hz) for calcite drilling fluid, according to certain embodiments.

FIG. 11B is a graphical representation illustrating exemplary plots of phase shift measurements at 7 time periods for lower channel 3 over a broad frequency range (0.1-10,000 Hz) for calcite drilling fluid, according to certain embodiments.

FIG. 12A is a graphical representation illustrating exemplary plots of real conductivity measurements at 7 time periods for upper channel 1 over a broad frequency range (0.1-10,000 Hz) for calcite drilling fluid, according to certain embodiments.

FIG. 12B is a graphical representation illustrating exemplary plots of real conductivity measurements at 7 time periods for lower channel 3 over a broad frequency range (0.1-10,000 Hz) for calcite drilling fluid, according to certain embodiments.

FIG. 13A is a graphical representation illustrating exemplary plots of imaginary conductivity measurements at 7 time periods for upper channel 1 over a broad frequency range (0.1-10,000 Hz) for calcite drilling fluid, according to certain embodiments.

FIG. 13B is a graphical representation illustrating exemplary plots of imaginary conductivity measurements at 7 time periods for lower channel 3 over a broad frequency range (0.1-10,000 Hz) for calcite drilling fluid, according to certain embodiments.

FIG. 14A is a graphical representation illustrating exemplary plots of impedance measurements at 7 time periods for upper channel 1 over a broad frequency range (0.1-10,000 Hz) for calcite drilling fluid, according to certain embodiments.

FIG. 14B is a graphical representation illustrating exemplary plots of impedance measurements at 7 time periods for lower channel 3 over a broad frequency range (0.1-10,000 Hz) for calcite drilling fluid, according to certain embodiments.

FIG. 15A is a graphical representation illustrating comparative analysis of impedance over time in channel 3 for barite drilling fluid, according to certain embodiments.

FIG. 15B is a graphical representation illustrating comparative analysis of impedance over time in channel 3 for calcite drilling fluid, according to certain embodiments.

FIG. 16A is a graphical representation illustrating comparative analysis of impedance vs. density in channel 3 for barite drilling fluid, according to certain embodiments.

FIG. 16B is a graphical representation illustrating comparative analysis of impedance vs. density in channel 3 for calcite drilling fluid, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

In recent years, the geophysical technique known as spectral induced polarization (SIP) has gained prominence for laboratory sample characterization [See: Ghorbani, A., Cosenza, P., Revil, A., Zamora, M., Schmutz, M., Florsch, N., & Jougnot, D. (2009)—Non-invasive monitoring of water content and textural changes in clay-rocks using spectral induced polarization: A laboratory investigation. Applied Clay Science, 43(3-4), 493-502; Izumoto, S. (2021)—Spectral induced polarization of calcite precipitation in porous media. Thesis; Okay, G., Leroy, P., Ghorbani, A., Cosenza, P., Camerlynck, C., Cabrera, J., Florsch, N., & Revil, A. (2014)—Spectral induced polarization of clay-sand mixtures: Experiments and modeling. Geophysics, 79(6), E353-E375; Revil, A., Schmutz, M., & Batzle, M. L. (2011)—Influence of oil wettability upon spectral induced polarization of oil-bearing sands. Geophysics, 76(5), 2-7; Skold, M., Revil, A., & Vaudelet, P. (2011)—The pH dependence of spectral induced polarization of silica sands: Experiment and modeling. Geophysical Research Letters, 38(12), 1-6, each incorporated herein by reference in its entirety]. SIP operates by measuring the low-frequency electric complex conductivity of materials, creating distinctive spectral signatures that can identify specific attributes. Although SIP has been primarily researched in controlled environments, with only a few applications in the field, its effectiveness for shallow subsurface and contaminated site characterization is well-documented. Uses of SIP include detecting both organic and inorganic pollutants, tracking processes such as microbial-induced calcite precipitation, and monitoring bioremediation efforts, particularly those involving hydrocarbons [See: Justin, Dru, Andrew, Sai, Danielle, & Fortin. (2019)—The mitigation of deep depth wellbore leakage using microbially induced carbonate precipitation (MICP). Geoconvention; Kirmizakis, P. (2016)—Laboratory scale application of spectral induced polarization (SIP) method for environmental monitoring. Thesis; Kirmizakis, P., Kalderis, D., Ntarlagiannis, D., & Soupios, P. (2020)—Preliminary assessment on the application of biochar and spectral-induced polarization for wastewater treatment. Near Surface Geophysics, 18(2), 109122; Kirmizakis, P., Tawabini, B., Siddiq, O. M., Kalderis, D., Ntarlagiannis, D., & Soupios, P. (2022)—Adsorption of Arsenic on Fe-Modified Biochar and Monitoring Using Spectral Induced Polarization. Water (Switzerland), 14(4), 1-16; Mellage, A., Smeaton, C. M., Furman, A., Atekwana, E. A., Rezanezhad, F., & van Cappellen, P. (2018)—Linking Spectral Induced Polarization (SIP) and Subsurface Microbial Processes: Results from Sand Column Incubation Experiments. Environmental Science and Technology, 52(4), 2081-2090; Ntarlagiannis, D., Kirmizakis, P., Kalderis, D., & Soupios, P. (2016)—Using the spectral induced polarization method to assess biochar performance as a remediation agent. AGU Fall Meeting 2016; Ntarlagiannis, D., Kalderis, D., & Soupios, P. (2020)—Geophysical Methods for Contaminant Management: The Case for Biochar. Fast TIMES, 25(2), 80-90; Personna, Y. R., Ntarlagiannis, D., Slater, L., Yee, N., O'Brien, M., & Hubbard, S. (2008)—Spectral induced polarization and electrodic potential monitoring of microbially mediated iron sulfide transformations. Journal of Geophysical Research: Biogeosciences, 113(2), 1-13; Siddiq, O. M., Tawabini, B. S., Soupios, P., & Ntarlagiannis, D. (2021)—Removal of arsenic from contaminated groundwater using biochar: a technical review. In International Journal of Environmental Science and Technology, each incorporated herein by reference in its entirety]. The method is also adept at determining concentrations of Fe(II) and the precipitation of metal sulfides, thanks to the direct correlation between SIP responses and polarizable targets [See: Ntarlagiannis, D., Williams, K. H., Slater, L., & Hubbard, S. (2005)—Low-frequency electrical response to microbial induced sulfide precipitation. Journal of Geophysical Research: Biogeosciences, 110(G2); Ntarlagiannis, D., Doherty, R., & Williams, K. H. (2010)—Spectral induced polarization signatures of abiotic FeS precipitation. Geophysics, 75(4), incorporated herein by reference in their entirety]. The potential applications of SIP extend beyond environmental studies, venturing into the oil and gas sector. Investigations have utilized SIP for analyzing oil wettability, enhancing oil recovery through microbial processes (EOR).

Aspects of the present disclosure are directed to a method for detecting and monitoring the sag value of weighting materials in drilling fluids within boreholes. The method of the present disclosure employs the spectral induced polarization (SIP) technique to inject alternating current into the drilling fluid, measure real-time conductivity and impedance, and calculate the densities at different elevations to determine the sag value. The present disclosure uses SIP as an early detection technique for tracking sag of weighting materials in the drilling fluids and monitoring this process over time. The present disclosure focuses on density variations, using the SIP to determine even slight changes in the concentration of weighting materials. Traditional methods like the standard sag and high-angle sag tests often fail to yield quick results, but sensitivity of the SIP allows for rapid assessment. The present disclosure employs both barite and calcite, the most prevalent types of drilling fluids, to demonstrate capacity of the SIP to monitor material settlement under static conditions. Real conductivity and impedance measurements are highly sensitive techniques that can detect even minor changes in the drilling fluid composition. Operators (drilling engineers) can closely monitor these parameters to identify sag in its nascent stages, allowing timely corrective actions. This early detection is crucial for preventing issues like stuck pipes or wellbore instability, which can lead to costly delays and accidents. The present disclosure allows for in-situ measurements without the need to extract fluid samples, thereby offering a more efficient and less intrusive means of monitoring drilling fluid integrity.

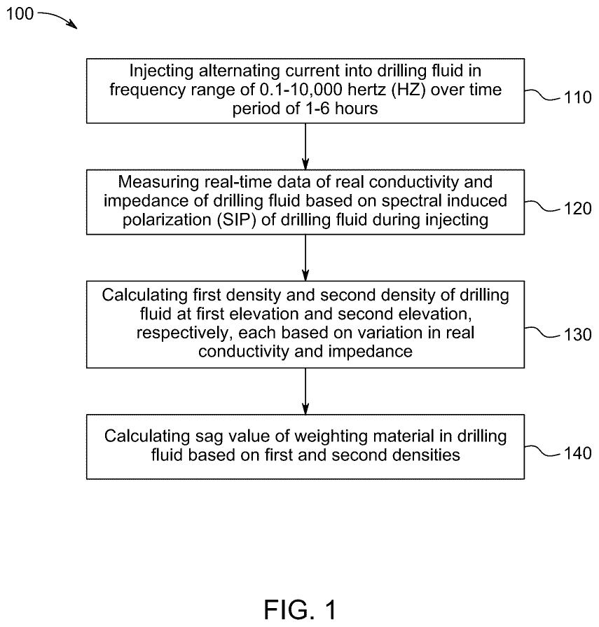

Referring to FIG. 1, illustrated is an exemplary flowchart of a method (as represented by reference numeral 100) of detecting a sag value of a weighting material in a drilling fluid in a borehole, in accordance with embodiments of the present disclosure. The method 100 utilizes the principles of spectral induced polarization (SIP) to assess changes in the electrical properties of the drilling fluid, which are indicative of the concentration and distribution of the weighting material particles. By measuring parameters such as real conductivity and impedance across a defined frequency range, the present method 100 enables the calculation of density variations at different elevations within the borehole. These calculations are used for determining the sag value of the weighting material, providing a direct correlation between the electrical properties of the drilling fluid and the concentration of particles therein. The capability to perform these measurements in real-time and without the necessity of extracting the drilling fluid from the borehole provide a more efficient, less intrusive means of maintaining the integrity of drilling fluids. Through the application of the present method 100, drilling engineers are equipped with timely and accurate data on the condition of the drilling fluid, facilitating immediate adjustments to mitigate the effects of weighting material sag and ensure the continuous stability of the wellbore.

It may be understood that the sag factor increases when solid particles settle in the drilling fluid, indicating poor fluid stability. As the sag factor increases, the concentration of solids in the drilling fluid rises, affecting its electrical conductivity. An increase in the sag factor leads to a decrease in the real (electrolytic) conductivity of the drilling fluid. This is because the settled solids create a barrier, hindering the flow of electric current through the drilling fluid. Real conductivity measurements can, therefore, indirectly indicate the sag factor by detecting changes in the ability of the drilling fluid to conduct electricity. Similarly, an increase in the sag factor results in a higher concentration of solids in the drilling fluid, leading to an increase in impedance. Impedance, which includes both resistance and reactance, reflects the overall hindrance to the flow of electric current. As the sag factor increases, the settled solids create resistance and alter the dielectric properties of the drilling fluid, contributing to higher impedance values. Additionally, barite and calcite are standard components of drilling fluids, each with unique properties. Real conductivity and impedance measurements enable the clear differentiation between these fluids. This differentiation is important because the response of different fluids to sag might vary.

At step 110, the method 100 includes injecting an alternating current into the drilling fluid in a frequency range of 0.1-10,000 hertz (Hz) over a time period of 1-6 hours. That is, the process begins with the injection of the alternating current into the drilling fluid, which is contained within the borehole. This injection of alternating current is conducted within a specified frequency range, e.g., from 0.1 hertz (Hz) to 10,000 Hz. Such selected range provides a broad spectrum of frequencies to ensure a comprehensive assessment of electrical properties of the drilling fluid, which, in turn, are influenced by the presence and behavior of weighting material particles within the drilling fluid. Further, the duration of the alternating current injection is controlled, extending over a time period ranging from 1 to 6 hours. This extended time frame captures the dynamic changes in the electrical properties of the drilling fluid, which may occur as the weighting materials undergo settling or sagging. By maintaining the injection of alternating current for this duration, the method 100 ensures that sufficient data are gathered to accurately reflect the state of the drilling fluid at various stages of the drilling process. The injection of alternating current serves as a foundational step in the method 100, enabling the subsequent measurement of real conductivity and impedance of the drilling fluid. These measurements allow for analyzing the SIP response of the drilling fluid, which is directly related to the concentration and distribution of the weighting material particles.

In an embodiment, an amplitude of the alternating current is in a range of 1-10 volts (V). This amplitude range is selected to ensure the correct induction of the SIP response within the drilling fluid without compromising integrity of the drilling fluid or the operational parameters of the drilling process. The controlled amplitude facilitates the effective propagation of the alternating current through the drilling fluid, permitting the accurate measurement of electrical properties thereof, which are indicative of the sagging behavior of the weighting materials. Further, in an embodiment, the frequency range is 1000-10,000 Hz, and the time period is in a range of from 1-3 hours. The narrowed range of 1000-10,000 Hz is chosen based on its effectiveness in producing a noticeable SIP response from the drilling fluid, particularly in relation to the detection of variations in the concentration and distribution of the weighting material particles. Operating within this frequency spectrum allows for a focused analysis of electrical characteristics of the drilling fluid, enhancing the sensitivity and accuracy of the sag detection process. Further, the duration over which the alternating current is injected into the drilling fluid being confined to a range of 1-3 hours represents a window for monitoring the dynamic changes in properties of the drilling fluid that may occur due to the settling of weighting materials. A shorter duration within this range may be sufficient for rapidly changing conditions or when initial signs of sagging are detected, whereas extending the injection period up to 3 hours allows for a more comprehensive assessment of stability of the drilling fluid over time. By defining this time frame, the method 100 ensures a balanced approach that accommodates the need for timely data acquisition while providing a thorough evaluation of the sagging phenomenon.

At step 120, the method 100 includes measuring real-time data of a real conductivity and an impedance of the drilling fluid based on a spectral induced polarization (SIP) of the drilling fluid during the injecting. The measurement of real-time data includes the acquisition of values pertaining to the real conductivity and the impedance of the drilling fluid, and is conducted concurrently with the injection of the alternating current into the drilling fluid. Herein, the SIP technique enables the detection of electrical property variations within the drilling fluid, which are reflective of changes in the concentration and distribution of the weighting material particles. As used herein, the real conductivity refers to the ability of the drilling fluid to conduct electrical current in its real component, excluding the effects of capacitive and inductive reactance. The measurement of real conductivity provides insight into the electrolytic behavior of the drilling fluid, which is directly influenced by the presence and behavior of the suspended weighting material particles. Variations in real conductivity are indicative of changes in composition and structure of the drilling fluid, such as those occurring due to the sagging of weighting materials. Further, the impedance refers to the total opposition offered by the drilling fluid to the flow of the injected alternating current. The impedance integrates both resistive and reactive components (capacitive and inductive), providing an assessment of electrical resistance of the drilling fluid. The measurement of impedance is particularly sensitive to the distribution and concentration of the weighting material particles within the drilling fluid. As such, changes in impedance values serve as a reliable indicator of the onset and progression of sagging within the drilling fluid. The concurrent measurement of the real conductivity and the impedance during the injection of alternating current allows for the continuous monitoring of electrical properties of the drilling fluid. This real-time data acquisition allows for the early detection of weighting material sag, enabling timely interventions to adjust composition or operational parameters of the drilling fluid, thereby maintaining the integrity and stability of the wellbore.

In some embodiments, the method 100 further includes measuring a phase shift and an imaginary conductivity of the drilling fluid at different elevations of the borehole. That is, an additional aspect of real-time data measurement involves the assessment of the phase shift and the imaginary conductivity of the drilling fluid. These measurements are conducted at various elevations within the borehole, providing a vertical profile of electrical properties of the drilling fluid and their variation with depth. This approach to data collection allows for understanding the spatial distribution of weighting material sag within the borehole, which may vary significantly from one section to another due to differences in hydrostatic pressure, temperature, and other wellbore conditions. The phase shift, in this context, refers to the lag between the injected alternating current (voltage) and the resulting current through the drilling fluid, expressed in degrees. This parameter provides insight into the capacitive behavior of the drilling fluid. Variations in the phase shift at different borehole elevations can indicate changes in composition of the drilling fluid, particularly the distribution of the weighting material particles, which can affect capacitive properties of the drilling fluid. Imaginary conductivity, on the other hand, represents the component of conductivity of the drilling fluid that is out of phase with the voltage applied by the alternating current. The imaginary conductivity is directly related to the phase shift and provides a measure of ability of the drilling fluid to store electrical energy temporarily. Like the phase shift, variations in imaginary conductivity at different borehole elevations can provide information about the sagging behavior of the weighting materials. An increase in imaginary conductivity could indicate a higher concentration of particles in suspension, whereas a decrease may suggest particle settling or sagging.

At step 130, the method 100 includes calculating a first density and a second density of the drilling fluid at a first elevation and a second elevation, respectively, each based on a variation in the real conductivity and the impedance. These calculations are based on observed variations in the real conductivity and the impedance of the drilling fluid, which are indicative of changes in composition and structure of the drilling fluid, particularly in relation to the concentration and distribution of weighting material particles. The process commences with the measurement of the real conductivity and the impedance at the first elevation and the second elevation, which are chosen to capture the vertical variation in properties of the drilling fluid for understanding the extent and impact of weighting material sag within the borehole. Upon obtaining the measurements of the real conductivity and the impedance at these elevations, the process involves analyzing these data to determine the corresponding densities of the drilling fluid. This analysis uses the direct relationship between the electrical properties of the drilling fluid (real conductivity and impedance) and its density. Variations in real conductivity and impedance reflect changes in composition of the drilling fluid, such as the addition or settling of weighting material particles, which in turn affect density of the drilling fluid. It may be contemplated that the calculation of the first density and the second density is performed using predefined models or equations that relate the measured electrical properties (real conductivity and impedance) to the density of the drilling fluid. These approaches are based on empirical data and theoretical understanding of the behavior of drilling fluids under various conditions. By calculating the densities at two different elevations, the method 100 provides insights into the vertical distribution of weighting materials within the drilling fluid. A significant difference between the first and second densities may indicate the occurrence of sagging, with a higher density at a lower elevation suggesting the settling of weighting material particles.

Herein, the variation in the real conductivity and the impedance directly relates to changes in a concentration of particles of the weighting material in the drilling fluid. That is, there is a direct correlation between the variation in the real conductivity and the impedance of the drilling fluid and the changes in the concentration of particles of the weighting material within the drilling fluid. This relationship enables detecting and monitoring of the sag value of the weighting materials, as it provides a measurable indicator of the distribution and behavior of these particles within the drilling fluid. The real conductivity, which measures the ability of the drilling fluid to conduct electrical current without the influence of capacitive or inductive properties, is sensitive to the ionic concentration and mobility within the drilling fluid. An increase in the concentration of particles typically leads to a decrease in real conductivity, as the suspended particles can disrupt the flow of ions, reducing overall conductive capacity of the drilling fluid. Conversely, the impedance, which includes both the resistive and reactive components of opposition of the drilling fluid to electrical current, is influenced by the physical and electrical properties of the drilling fluid. An increase in the concentration of weighting material particles increases impedance of the drilling fluid due to the added resistance and alteration in the dielectric properties introduced by the weighting particles. The present method 100 uses these relationships by continuously monitoring the real conductivity and impedance of the drilling fluid at various elevations within the borehole. Variations in these electrical properties helps in determining changes in the concentration of weighting material particles, enabling the detection of sagging phenomena in real-time. Further, by quantifying these variations, the method 100 facilitates the calculation of density changes in the drilling fluid, which are directly related to the sag value of the weighting materials.

In particular, in the present implementation, the method 100 includes calculating the concentration of the particles of the weighting material in the drilling fluid based on the real conductivity and the impedance. It may be understood that the relationship between these electrical properties and the concentration of weighting material particles is based on the principle that the presence and distribution of these particles within the drilling fluid influence its ability to conduct electricity and its overall electrical resistance. To perform this calculation, the method 100 utilizes established models or equations that relate the real conductivity and impedance of the drilling fluid to the concentration of suspended particles. Through electrical resistance vs. density for CaCO₃ and BaSO₄, a linear relationship between impedance and density exists as evidenced by the linear fits applied to the data. The corresponding equations are linear models that obtained, e.g., through linear regression analysis which is a statistical method used to find the best-fitting line through a set of data points. Linear regression analysis helps to model the relationship between a scalar response (or dependent variable, like impedance) and one or more explanatory variables (or independent variables, like density).

These relationships are derived from empirical observations and theoretical understandings of how particulate matter within a fluid affects its electrical properties. In the context of converting electrical resistance to particle concentrations, the almost linear relationship between impedance and density implies that as the concentration of particles in a medium increases, so does the medium's density, which, in turn, affects the impedance. The relationship is based on the principle that the presence of particles in a solution or suspension can disrupt the flow of electric current, thereby increasing the electrical impedance. For instance, an increase in the concentration of weighting material particles typically leads to a decrease in real conductivity due to the particles' disruption of the ionic pathways within the drilling fluid. Simultaneously, the impedance of the drilling fluid tends to increase with higher particle concentrations because the suspended particles introduce additional resistance and alter the dielectric properties of the drilling fluid.

At step 140, the method 100 includes calculating the sag value of the weighting material in the drilling fluid based on the first and second densities. As discussed, the first and second densities of the drilling fluid are reflective of the concentration of particles of the weighting material at different depths. To calculate the sag value, the method 100 employs a comparative analysis of the first and second densities. The sag value quantifies the degree of separation or settling of the weighting materials within the borehole. A higher density at the lower elevation (second density) compared to the upper elevation (first density) typically indicates that sagging has occurred, with weighting material particles settling towards the bottom of the borehole due to gravitational forces and fluid dynamics. The specific formula used for calculating the sag value generally involves a function of the difference or ratio between the first and second densities (such as equation (1) provided in the preceding paragraphs). This calculation may also take into consideration other factors such as the depth of the borehole, the specific gravity of the weighting material, and properties of the drilling fluid to provide a more accurate assessment of the sag condition. The upper elevation (first density) measurement is preferably taken from the drilling mud as it exits the mud pump and before it enters the wellbore/system. It represents the initial or baseline density of the fluid, free from any influence of the borehole environment. This baseline is used for understanding the fluid's behavior under undisturbed conditions. After the mud circulates through the wellbore, interacting with formation pressures, temperatures, and contaminants, its density is measured again as it returns to the surface. This is the second density, and it is generally higher if there has been settling of weighting materials or absorption of formation materials. A discrepancy between these two densities can indicate various phenomena, including sag, where the heavier particles (like barite used to increase mud density) settle due to gravitational forces. If the second density is significantly higher than the first, it suggests that sagging has occurred, impacting the fluid's effectiveness and potentially leading to drilling complications like stuck pipes or poor hole cleaning. The units for sag value can vary; it might be expressed in pounds per gallon per foot (ppg/ft), for example, or in dimensionless units if a ratio is used instead. By calculating the sag value, the present method 100 provides a measurable parameter that can be used to evaluate the performance and stability of the drilling fluid. A higher sag value may prompt the need for corrective actions, such as adjusting composition of the drilling fluid, introducing additives to enhance viscosity or particle suspension, or modifying operational parameters to mitigate the effects of sagging. Conversely, a low sag value suggests good stability and homogeneous density throughout the fluid column. Understanding the sag value helps in adjusting the mud formulation or circulation practices to reduce settling. Techniques might include increasing the viscosity of the mud, improving the suspension properties of the fluid, or modifying the pumping schedule to ensure more consistent fluid properties.

In the present implementation, the borehole has a depth of less than 1 km and the injecting occurs concurrently in a top, a middle, and a bottom of the borehole. That is, the borehole, in which the drilling fluid is contained and monitored, having a depth of less than 1 kilometer is considered for implementation of the method 100 of the present disclosure. Within this borehole, the method 100 involves the concurrent injection of the alternating current into the drilling fluid at three distinct elevations: the top, the middle, and the bottom. This approach to alternating current injection provide an overview of electrical properties of the drilling fluid and their variation along the vertical extent of the borehole. The concurrent injection of the alternating current at the top, middle, and bottom of the borehole allows for the simultaneous assessment of electrical properties of the drilling fluid at these points. By targeting three different elevations, the method 100 ensures that the data collected on real conductivity, impedance, phase shift, and imaginary conductivity reflect the conditions across the entire depth of the borehole, rather than being limited to a single point. This is particularly important in the context of weighting material sag, as the phenomenon can vary significantly with depth due to factors such as hydrostatic pressure gradients, temperature variations, and the mechanical disturbances induced by drilling operations.

In the present method 100, the variation in the real conductivity and the impedance of the drilling fluid are quantified to provide diagnostic information regarding the sagging of weighting materials within the fluid. In an embodiment, the variation in the real conductivity is 1-20 μS/cm, preferably 5-15 μS/cm or about 10 μS/cm, compared to an initial real conductivity. This variation reflects changes in ionic makeup of the drilling fluid due to alterations in the concentration of the weighting material particles, as they either suspend uniformly or settle. Further, the variation in the impedance is 1-20 ohms, preferably 5-15 ohms or about 10 ohms, compared to an initial impedance. This parameter provides insight into the total resistance the fluid offers to the flow of electrical current, integrating both resistive and capacitive components of the drilling fluid. The specified range of variation in impedance is indicative of the settling behavior of the weighting materials, with increased impedance often signaling a higher degree of particle settlement and resulting in heterogeneity within the drilling fluid. It may be understood that by comparing the variations in the real conductivity and the impedance to their initial values, the method 100 provides a dynamic and sensitive approach to monitoring condition of the drilling fluid, enabling timely adjustments to the drilling operation to maintain fluid integrity and wellbore stability.

Further, in the present method 100, the drilling fluid is characterized by a specific salinity range, which is a measure of the concentration of dissolved salts within the drilling fluid. In an embodiment, the drilling fluid has a salinity of 1 to 1000 ppt, preferably 10-500 ppt, 50-250 ppt or about 100-200 ppt. This salinity range is broad to accommodate various drilling scenarios and fluid formulations that may be employed in different geological conditions. Salinity can affect both the real conductivity and impedance of the drilling fluid; therefore, it is defined to regulate its impact on the electrical property measurements used for sag detection. Additionally, it may be noted that temperature may have a significant influence on the viscosity, density, and electrical properties of the drilling fluid, as well as the solubility and stability of the weighting materials. High temperatures can accelerate the sagging of weighting materials due to changes in the properties and the reduction of viscosity of the drilling fluid, which can affect the suspension of solids. For purposes of the present method 100, the drilling fluid is in a reservoir and the reservoir has a temperature of 100-300° C. preferably 150-250° C. or about 200° C. Herein, the method 100 considers the environmental conditions in which the drilling fluid is utilized, specifically the temperature range of the reservoir in which the drilling fluid is located. The temperature of the reservoir is accounted for within a range of 100 to 300 degrees Celsius (° C.). This temperature range is indicative of the harsh conditions that drilling fluids may encounter in deep and high-temperature drilling environments, such as geothermal reservoirs or deep-terrestrial hydrocarbon reservoirs.

Furthermore, in the present method 100, the location and the conditions of the drilling fluid under which the injecting and measuring of electrical properties are performed are specified to ensure accurate and reliable data collection. In one implementation, the drilling fluid is in a subterranean borehole having a depth of at least 500 m during the injecting and the measuring. The depth parameter ensures that the method 100 is tested and applicable in environments that are representative of many operational drilling conditions, where pressure, temperature, and wellbore stability present unique challenges. In another implementation, the drilling fluid is in a horizontal well during the injecting and the measuring. Horizontal wells present unique challenges compared to vertical wells, including higher tendencies for sag due to the increased surface area available for solids to settle and differences in hydrostatic pressures. The method 100 being designed to function in horizontal wells demonstrates its versatility and capacity to adapt to various drilling orientations. For example, measurements may be taken in a horizontal section, or plurality of horizontal sections, preferably 3-12 or 5-10 horizontal sections separated by at least 25 meters or 250-500 meters by packers.

In present implementations, the measuring is downhole in a well during the injecting and the measuring. That is, as per the present method 100, the measuring of the real conductivity and the impedance of drilling fluid is performed downhole within the well during the injecting of the alternating current. This downhole measurement captures real-time data on electrical properties of the drilling fluid in the actual drilling environment. Further, the method 100 does not comprise extracting the drilling fluid from the borehole for the measuring. Conducting these measurements in situ ensures that the data are not only representative of the well conditions but also that they are collected without the need for fluid extraction, which could disrupt the drilling process and introduce delays. This eliminates the need for physical removal of fluid samples to conduct the analysis, which traditionally requires halting the drilling process and can introduce delays and complexities in the operational workflow.

### EXAMPLES

preparation of materials and samples, the application of the Portable Spectral Induced Polarization (SIP) technology in both laboratory and field settings, and the comprehensive test setup and procedure including precise formulation of the drilling fluids, the specific types and quantities of weighting materials used, and the conditions under which the samples are prepared to replicate the downhole environment. The SIP technology employed, the test setup and procedure are further described below according to specific configurations used for injecting the alternating current into the drilling fluid, the placement and role of electrodes, the temporal aspects of the testing period, and the methodology for capturing and interpreting the data generated.

### Materials and Sample Preparation

The drilling fluids utilizing barite and calcite were formulated according to the specific recipes detailed in Table 1, adhering to the conventional protocols for mixing drilling fluids. Deionized water was used to ensure the purity of the samples and to prevent any interference that could arise from the salinity or other elements in the water. This precaution is adopted because the spectral induced polarization (SIP) method is extremely sensitive to even minor impurities. Following preparation, the samples underwent density testing to validate their composition.

TABLE 1
Barite and calcite drilling fluid formula

| | | |
|---|---|---|
| Component | Weight % | Mixing time |

Barite fluid

| | | |
|---|---|---|
| XC Polymer | 0.19 | 20 minutes |
| Starch | 0.76 | 10 minutes |
| Barite | 53.00 | 10 minutes |
| Water | 46.04 | — |

Calcite fluid

| | | |
|---|---|---|
| Bentonite | 4 | 10 minutes |
| Calcite | 5 | 10 minutes |
| Water | 91 | — |

As discussed, the composition of the drilling fluid is an important aspect of the methodology, particularly concerning the weighting material. In an embodiment of the present disclosure, the drilling fluid comprises about 40-60 wt. % barite or about 50 wt. %, based on a total weight of the drilling fluid. That is, the drilling fluid includes about 40-60 weight percent (wt. %) barite, based on the total weight of the drilling fluid. This range helps to achieve the desired density necessary for wellbore stability and to counteract the formation pressures encountered during drilling operations. The drilling fluid may further be constituted of additional components for its rheological properties and stability. Herein, the drilling fluid further comprises 40-60 wt. %, or about 50 wt. %, water, 0.1-1 wt. %, or about 0.5 wt. %, of a polymer, and 0.1-1 wt. %, or about 0.5 wt. %, of a starch, each based on the total weight of the drilling fluid. The 40-60 wt. % water is integral for mixing of constituent particles and for cooling purposes. The drilling fluid also contains 0.1-1 wt. % of a polymer and 0.1-1 wt. % of a starch, each based on the total weight of the drilling fluid. These additives enhance viscosity and filtration properties of the drilling fluid, and also enhances its ability to suspend and carry solids. In the present embodiment, the first and second densities of the drilling fluid can be determined according to the equation d_{barite}=(Z+575.37)/381.02. Herein, d_{barite} denotes the first and second densities of the drilling fluid comprising barite, respectively; and Z denotes the first and second impedance of the drilling fluid, respectively. The equation provides a direct means to quantify density of the drilling fluid based on the impedance, which changes as barite particles settle, thus altering its overall electrical resistance.

In another embodiment, the drilling fluid comprises 1-10 wt. % calcite, based on a total weight of the drilling fluid. This concentration range of calcite, a different weighting material from barite, is selected to provide necessary density adjustments for the drilling fluid to maintain wellbore integrity while drilling through various geological formations. Herein, the drilling fluid further comprises 1-10 wt. %, preferably 2-8 wt. % or about 5 wt. %, bentonite and 80-98 wt. % water, each based on a total weight of the drilling fluid. Bentonite, a clay mineral, is incorporated for its thixotropic properties, which confer upon the drilling fluid the ability to carry cuttings effectively and ensure the stability of the borehole wall. The high percentage of water acts as the base fluid in which these solids are suspended, ensuring the fluidity and the transfer of hydrostatic pressure to the wellbore. In the present embodiment, the first and second densities of the drilling fluid can be determined according to the equation d_{calcite}=(Z+690.76)/902.16. Herein, d_{calcite} denotes the first and second densities of the drilling fluid comprising calcite, respectively; and Z denotes the first and second impedance of the drilling fluid, respectively. This equation allows for the calculation of density of the drilling fluid by relating it to the impedance, which reflects the resistance to electrical current flow altered by the presence of calcite particles. Such a calculation helps to evaluate behavior of the drilling fluid, specifically how calcite particles contribute to the overall density and potentially to the sag phenomenon.

### Portable (Lab/Field) Spectral Induced Polarization (PSIP) Unit

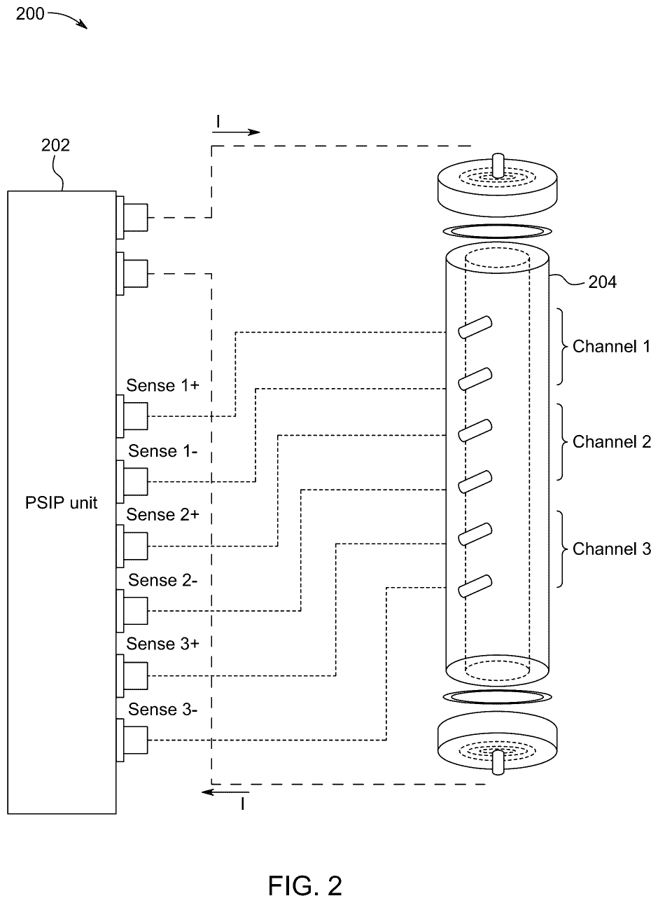

Referring to FIG. 2, illustrated is a system (as represented by reference numeral 200) for detecting a sag value of a weighting material in a drilling fluid in a borehole. The system includes a Portable Spectral Induced Polarization (PSIP) device 202 and a SIP measurement column 204. The PSIP device 202 is a multi-channel device which may be equipped with four channels for current application and 24 pairs of voltage sensing electrodes. The PSIP device 202 is capable of concurrently testing four distinct samples, as each current channel operates independently. For signal recording, the sample under examination is positioned within the SIP measurement column 204, which is a specially designed chamber that is outfitted with electrodes for both current injection and voltage sensing to capture the SIP signals.

In an example, as discussed, the measured parameters in the SIP survey are the phase shift (φ) and the magnitude of conductivity (|σ|) across a wide frequency range (1 mHz-20 kHz) of an alternating current. These parameters are then transformed into the real (σ′) and imaginary (σ″) components of the complex conductivity (σ*). The PSIP device 202 is equipped with the capability to adjust the shunt resistor value, which is essential when high current densities are involved. The shunt resistor ensures that a small, known fraction of the current passes through the ammeter, with selectable resistance values of 10, 100, 1000, and 10000 ohms to suit the specific needs of the sample under test. The method 100 involves recording these parameters and then applying them to calculate the real and imaginary parts of complex conductivity, utilizing the relationships defined in equations 2, 3, and 4 [Kimak, C., Ntarlagiannis, D., Slater, L. D., Atekwana, E. A., Beaver, C. L., Rossbach, S., Porter, A., & Ustra, A. (2019)—Geophysical Monitoring of Hydrocarbon Biodegradation in Highly Conductive Environments. Journal of Geophysical Research: Biogeosciences, 124(2), 353-366; Kirmizakis et al., 2020, incorporated herein by reference in their entirety] below.

$${σ}^{*} = {σ}^{′} + {σ}^{″} \qquad ( 2 )$$

$${σ}^{″} = \vert {σ}^{*} \vert sin ϕ \qquad ( 3 )$$

$${σ}^{′} = \vert {σ}^{*} \vert cos ϕ \qquad ( 4 )$$

In SIP surveys, the phase shift and imaginary conductivity serve as metrics for a sample's capacity to store electrical energy. Conversely, real conductivity and impedance, respectively quantify the overall ability of the sample to conduct electricity and its total resistance to electrical current, which includes both resistive and reactive components. In the present disclosure, real conductivity and impedance are the primary metrics of focus. Due to the intricate nature of the multi-component drilling fluids utilized, the link between density variations and electrical polarization may not be distinctly observable. Therefore, phase shift and imaginary conductivity play a supporting role, enhancing the qualitative interpretation of the real conductivity and impedance data.

### Test Setup and Procedure

The procedure for assessing sag and its verification consists of two fundamental steps: conducting SIP tests and confirming the results through density measurements. For the SIP tests, the present disclosure use a configuration of the SIP measurement column 204 featuring 340 ml columns. Within these SIP columns, potential electrodes are arranged into three segments based on their vertical position, with channels 1 and 3 designated for testing. The spacing between the centers of adjacent channels is 5 cm, and the gap between the topmost and bottom channels (1 and 3) is 12.5 cm. Fluid sampling ports are positioned directly across from each testing segment, on the opposite side of the column. These allow for the extraction of fluid from points directly corresponding to the testing channels, facilitating accurate density measurements to validate the SIP test results.

In the SIP surveys, the duration of the tests varied according to the type of fluid tested, with barite undergoing testing for 5.5 hours and calcite for 3 hours. This difference in testing periods is attributable to the distinct densities of each tested fluid. The tests were carried out in cycles at specific frequency points, which are detailed in Table 2.

TABLE 2
SIP test specifications for barite and calcite drilling fluids

| | | | | Shunt | | |
|---|---|---|---|---|---|---|
| | | | | Current | Loop | |
| | Freq. | Sampling | Amplitude | Resistor | time | Number |
| Fluid | (Hz) | steps | (V) | (Ohms) | (sec) | of loops |
| Barite | 0.1- | 6 logarithmic | 5 | 100 | 117 | 178 |
| | 10,000 | steps | | | | |
| Calcite | 100- | 3 logarithmic | 5 | 100 | 28 | 300 |
| | 10,000 | steps | | | | |

### Preliminary Sag and SIP Sample Testing

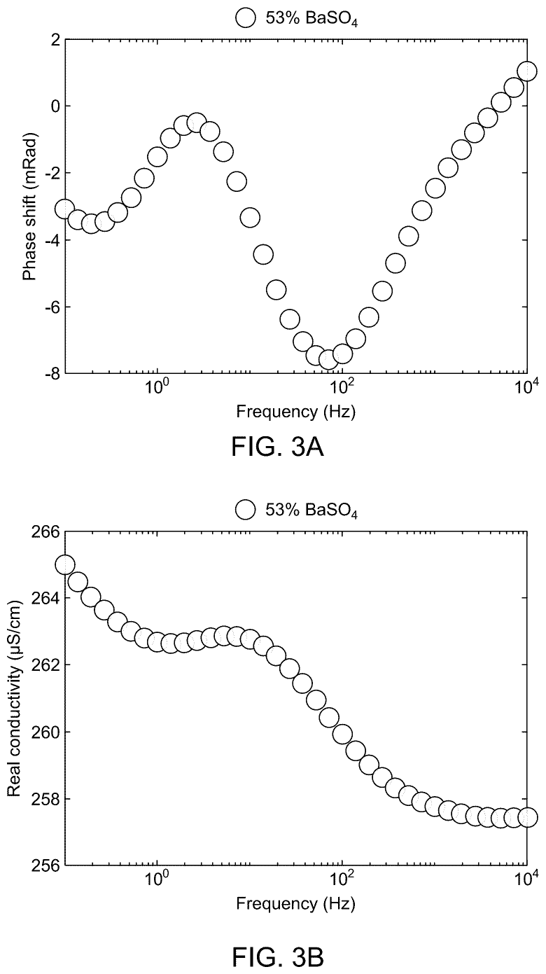

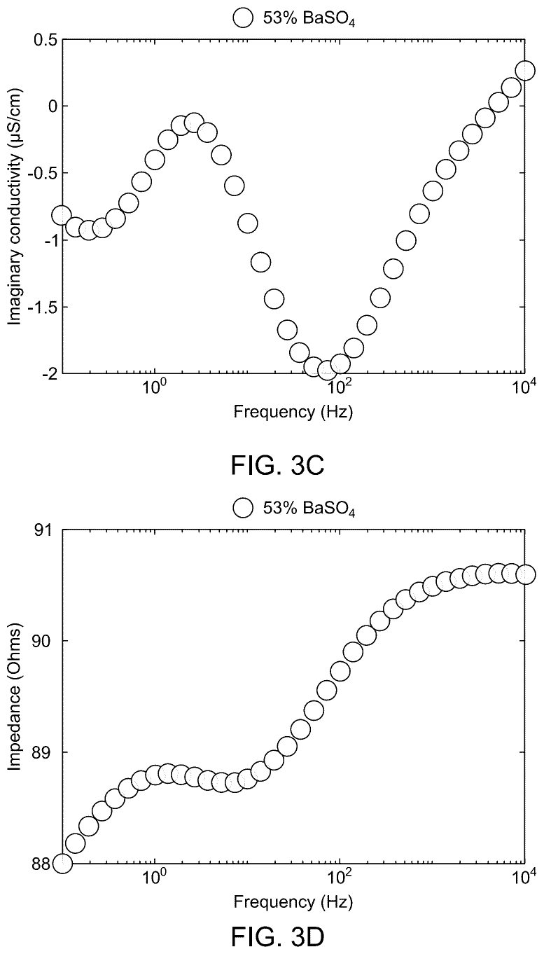

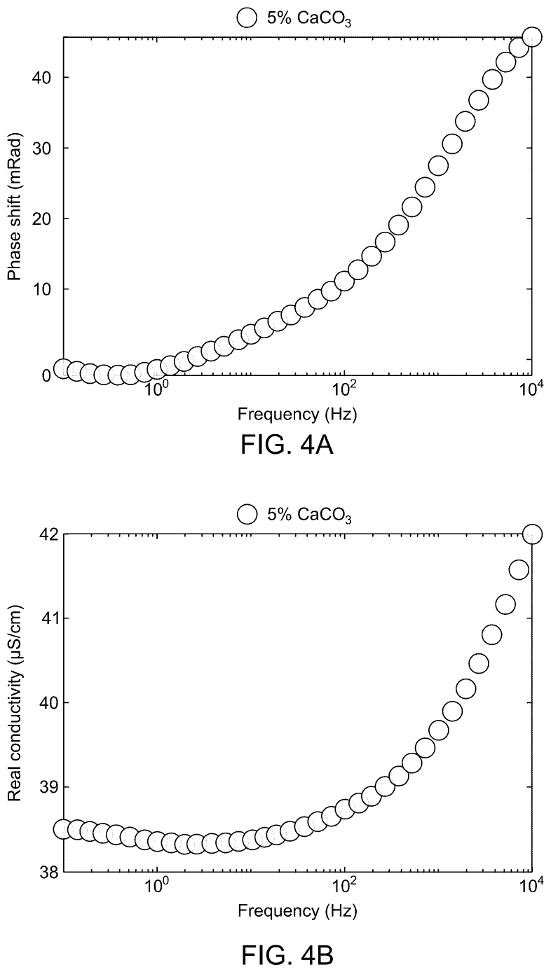

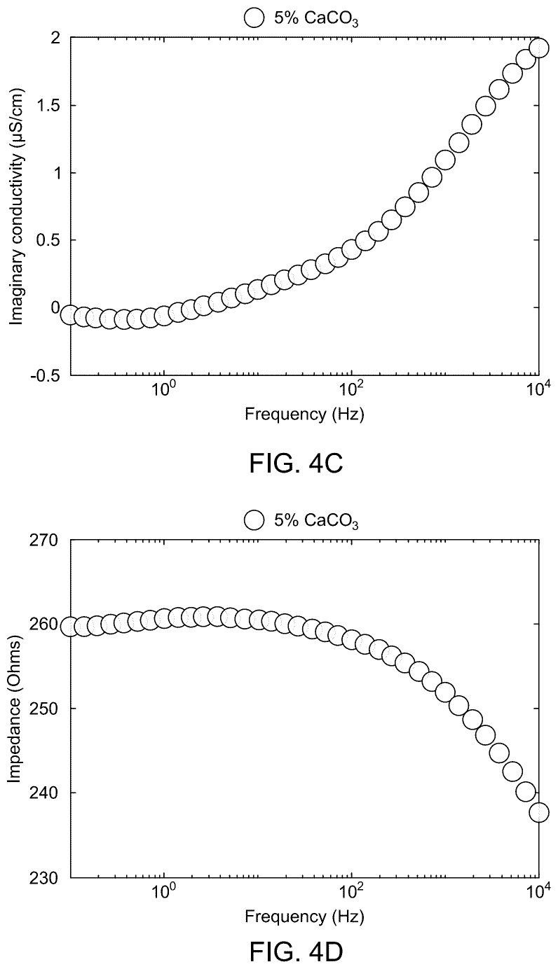

Before beginning the main experiments, full-spectrum SIP tests were performed on both barite and calcite. This is to comprehend their respective behaviors and to determine the most suitable frequency range and the acquisition time for the chosen frequencies. FIGS. 3A-3D are graphical representations illustrating exemplary plot(s) of phase shift, real conductivity, imaginary conductivity, and impedance, respectively, in SIP measurements over a broad frequency range (0.1-10,000 Hz) for barite drilling fluid; and FIGS. 4A-4D are graphical representations illustrating exemplary plot(s) of phase shift, real conductivity, imaginary conductivity, and impedance, respectively, in SIP measurements over a broad frequency range (0.1-10,000 Hz) for calcite drilling fluid. The results, illustrated in FIGS. 3A-3D and 4A-4D, reveal distinct SIP responses for barite and calcite across all signals. These responses indicate the unique characteristics of each material and their respective reactions to electrical currents. The differing chemical and electrical properties of barium sulfate (barite) and calcium carbonate (calcite) result in varied signal magnitudes. Specifically, barite exhibits a lower phase shift and imaginary conductivity but higher real conductivity and impedance compared to the calcite. This suggests a relatively higher electrical conductivity in barite.

The weight of the drilling fluid at the positions of the channels was assessed both before and after the test to determine the change in density resulting from sag. The data in Table 3 illustrate a decrease in density at the position of channels 1 and 3 for both samples, attributed to the settling of solid particles at base of the column. This settlement, indicating weighting material sag in both barite and calcite drilling fluids, is linked to the density variations. These differences in densities are further explored in the subsequent description, where they are correlated with time-variant changes in SIP signals. The percentage of settlement, calculated based on the time-related density changes (g/cc), is also detailed further in the description, comparing the initial (0 min) and final (180 min) states.

TABLE 3
Density evaluation with time for barite and calcite drilling fluids

| | Barite drilling fluid | | Calcite drilling fluid | |
|---|---|---|---|---|
| | Density channel 1 | Density channel 3 | Density channel 1 | Density channel 3 |
| Time (min) | (g/cc) | (g/cc) | (g/cc) | (g/cc) |
| 0 | 1.695 | 1.695 | 1.0032 | 1.0032 |
| 10 | 1.6127 | 1.695 | 1.0035 | 1.0129 |
| 40 | 1.611 | 1.7176 | 1.005 | 1.0161 |
| 60 | 1.6035 | 1.7431 | 1.0063 | 1.0165 |
| 90 | 1.6002 | 1.7719 | 1.0069 | 1.0185 |
| 120 | 1.599 | 1.7825 | 1.0069 | 1.019 |
| 180 | 1.587 | 1.7841 | 1.0072 | 1.02 |
| Difference (g/cc) | 0.108 | 0.0891 | 0.002 | 0.0168 |
| Difference (%) | 6.58 | 5.12 | 0.4 | 1.66 |

### SIP Signals Time Lapse at 10 kHz

The initial findings indicate that SIP signals exhibit peaks at higher frequencies, specifically in the 1000-10,000 Hz range, for both types of drilling fluids. Focusing on the 10 kHz frequency, where a significant average difference over time was noted, similar patterns were observed at other frequencies as well. The present implementation involved conducting SIP tests with barite and calcite drilling fluids, consisting of 178 loops of 117 seconds and 300 loops of 28 seconds, respectively. These tests aimed to monitor the settlement of weighting materials in two distinct channels: an upper channel (channel 1) and a lower channel (channel 3). Notably, the test results revealed discernible plot shifts between these two channels, attributed to slight variations in particle concentration over the test duration.

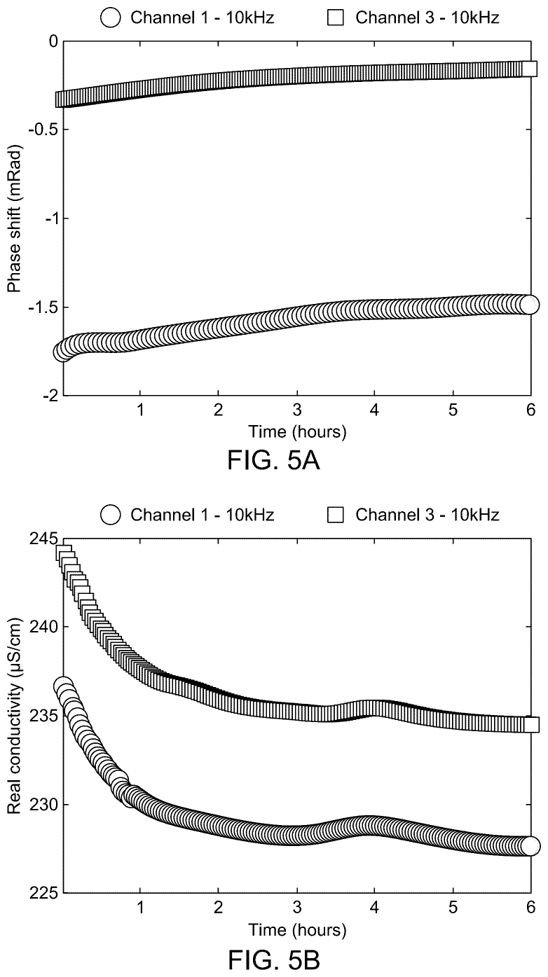

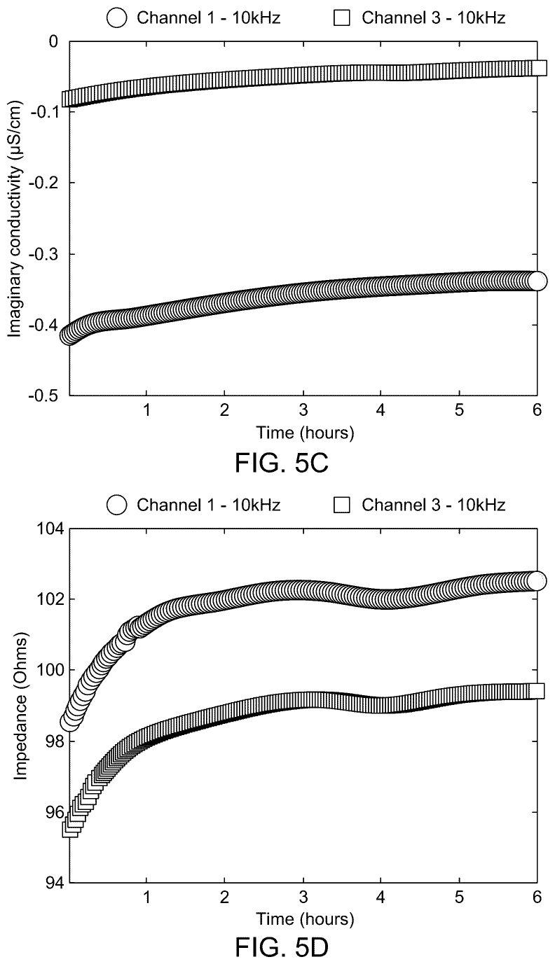

FIGS. 5A-5D are graphical representations illustrating exemplary plot(s) of timelapse of phase shift, real conductivity, imaginary conductivity, and impedance, respectively, in SIP signal measurements for barite drilling fluid at 10 kHz. As illustrated in FIGS. 5A and 5C, both the phase shift and the imaginary conductivity exhibited a marginal change over time in both channels. The phase shift, which is directly proportional to conductivity, tends to decrease with a lower solid concentration, reflecting the loss of solids that contribute to conductivity of the drilling fluid. Given that deionized water is the base fluid, a decrease in conductivity is expected with the loss of barite particles. However, these changes in phase shift and imaginary conductivity are not sufficiently indicative of the sedimentation process, as they are more sensitive to materials with high chargeability. Conversely, the real conductivity and the impedance emerged as more reliable indicators of particle loss, as evidenced by their dynamic changes over time, particularly in the first two hours, as shown in FIGS. 5B and 5D. The signal changes were most pronounced during this period, with a total variation of 11.5 μS/cm (microSiemens per centimeter) in real conductivity and 4.9 ohms in impedance. These measurements have inverse reactions to concentration changes, as impedance increases with a reduction in conductivity, while real conductivity decreases, reflecting its direct relation to electrical conductivity. The slowing of signal differences after the initial two hours suggests a decrease in particle migration.

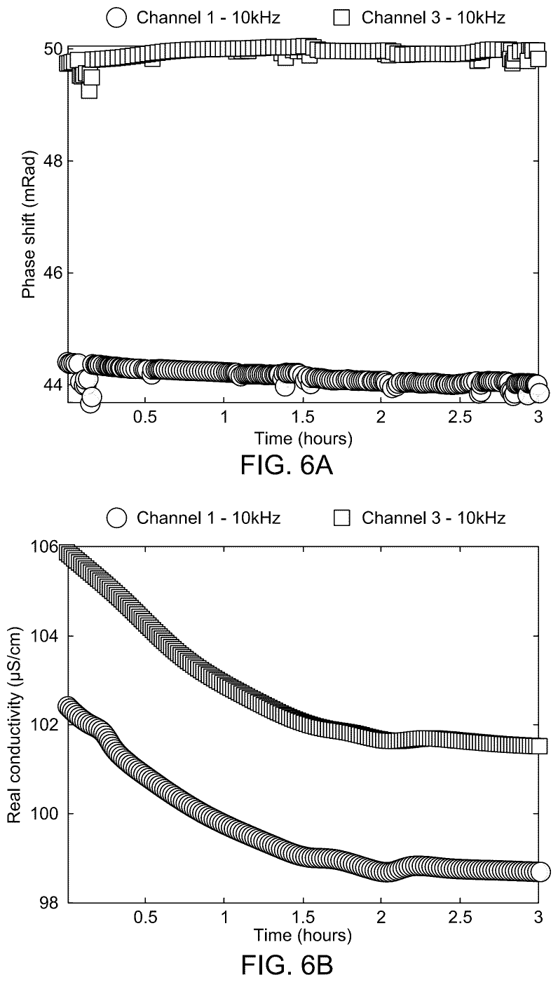

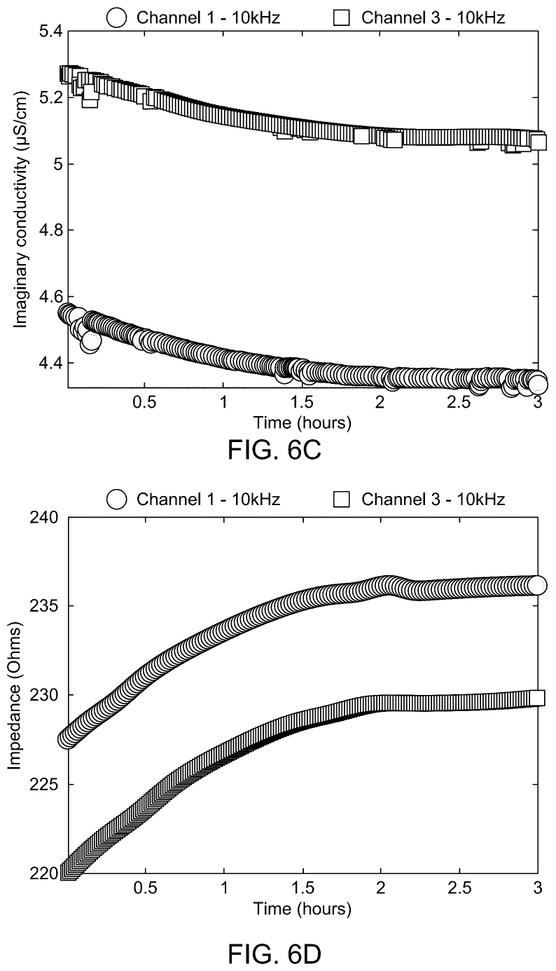

FIGS. 6A-6D are graphical representations illustrating exemplary plot(s) of timelapse of phase shift, real conductivity, imaginary conductivity, and impedance, respectively, in SIP signal measurements for calcite drilling fluid at 10 kHz. As depicted in FIGS. 6A and 6C, calcite drilling fluid exhibits a similar pattern to barite, with a decrease in conductivity due to the loss of solids. The responses of phase shift and imaginary conductivity in calcite also diminish over time, mirroring the decrease in conductivity. Notably, calcite shows a higher propensity for sag compared to barite, as evidenced by the greater dispersion observed between the two channels, which is attributable to the difference in concentration. However, these measures alone do not provide a comprehensive understanding of the ongoing phenomena. More revealing insights are found in the real conductivity and impedance plots of calcite, as depicted in FIGS. 6B and 6D, which tend to flatten earlier than those of the barite drilling fluid. These parameters demonstrate a more pronounced dispersion and provide more definitive signals, closely associated with changes in concentration. The average changes observed in calcite are 3.9 μS/cm for real conductivity and 9.5 ohms for impedance. These shifts distinctly illustrate the loss of particles, leading to a reduction in concentration and conductivity. This trend can be tracked effectively through the real conductivity and impedance plots of FIGS. 6B and 6D. The slowing of these effects around the 2-hour mark suggests a deceleration in the movement of particles towards bottom of the column. The changes in real conductivity and impedance over time for calcite drilling fluid are less pronounced than those in barite drilling fluid, primarily because calcite is inherently less conductive. Consequently, the reduction in real conductivity is smaller, as the particle loss in the channel contributes less significantly to the overall conductivity. However, the change in impedance is more marked, indicating an increase in resistance of the drilling fluid to electrical current flow. This increase occurs as the quantity of solids, which contribute to the colloidal conductive system, diminishes.

SIP Signals Behavior with Time

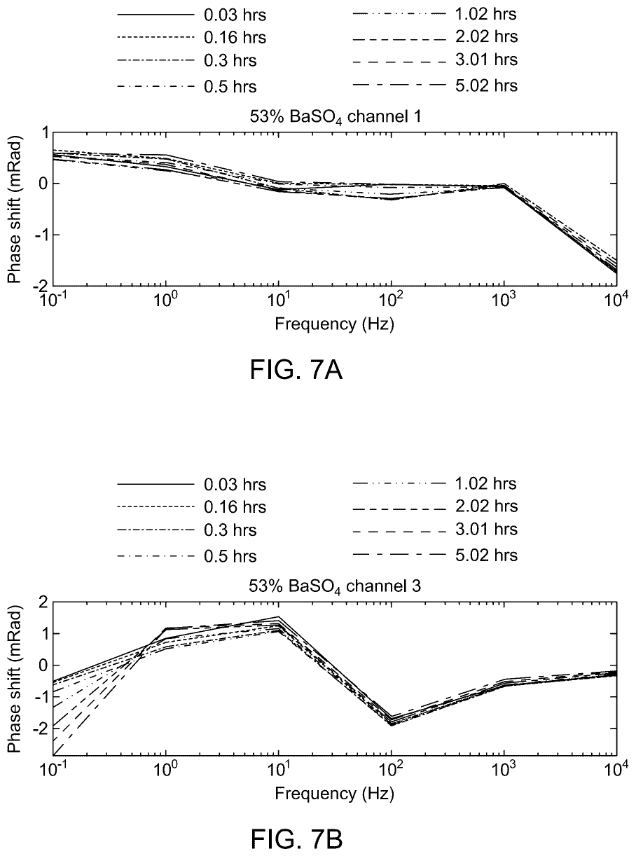

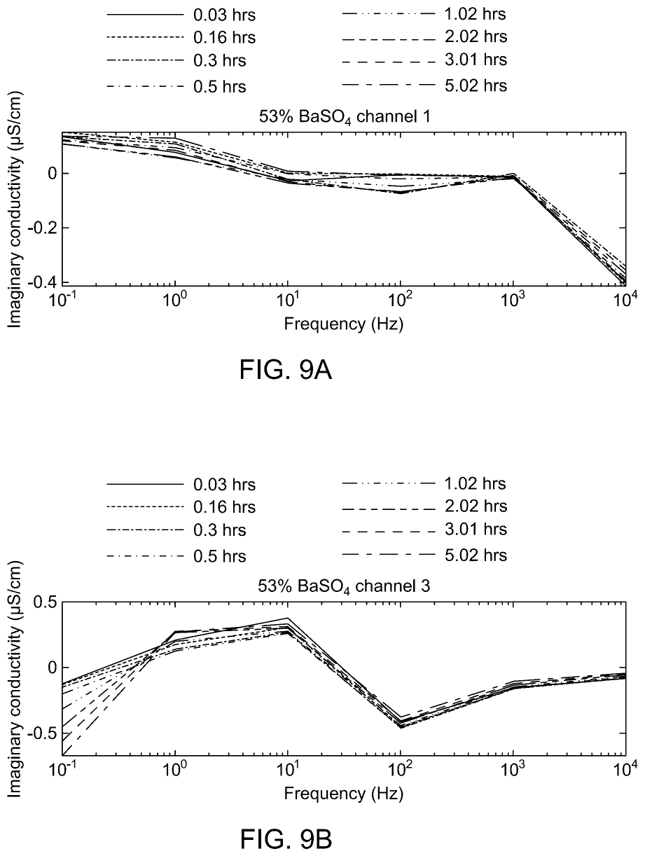

To comprehensively observe the changes in behavior over time, the full spectra for each testing interval were plotted for both barite and calcite drilling fluids. For the barite drilling fluid, data were collected at six selected frequencies, while for the calcite drilling fluid, three frequencies were analyzed. The specific testing times for the barite drilling fluid were set at 0.03, 0.16, 0.3, 0.5, 1.02, 2.02, 3.01, and 5.02 hours. For the calcite drilling fluid, the testing times were 0.01, 0.16, 0.3, 0.5, 1, 2, and 3 hours. In the case of the barite drilling fluid, both the phase shift and the imaginary conductivities (FIGS. 7A-7B and 9A-9B) exhibited only minor variations across different testing times. This limited dispersion can be attributed to the inherent characteristics of the samples. It is important to note that significant fluctuations in the phase shift and the imaginary conductivity values are not typically expected in non-metallic materials like barite and calcite, as these parameters are more indicative of the energy storage capabilities of metallic materials. Given that barite and calcite are not known for high energy storage properties, only subtle changes in the phase shift and the imaginary conductivities were anticipated. These minor alterations are not considered to provide a robust indication of particle settlement within the drilling fluids.

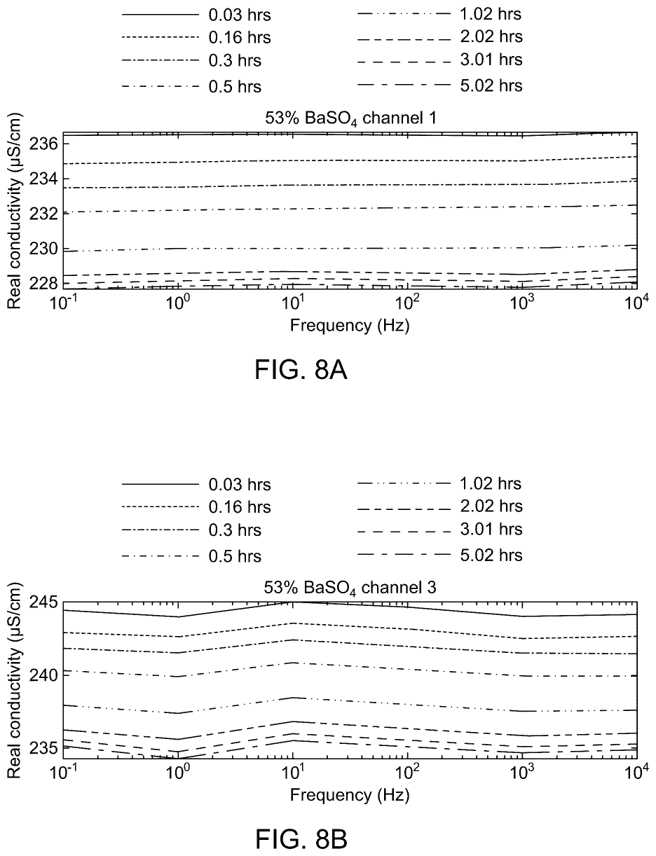

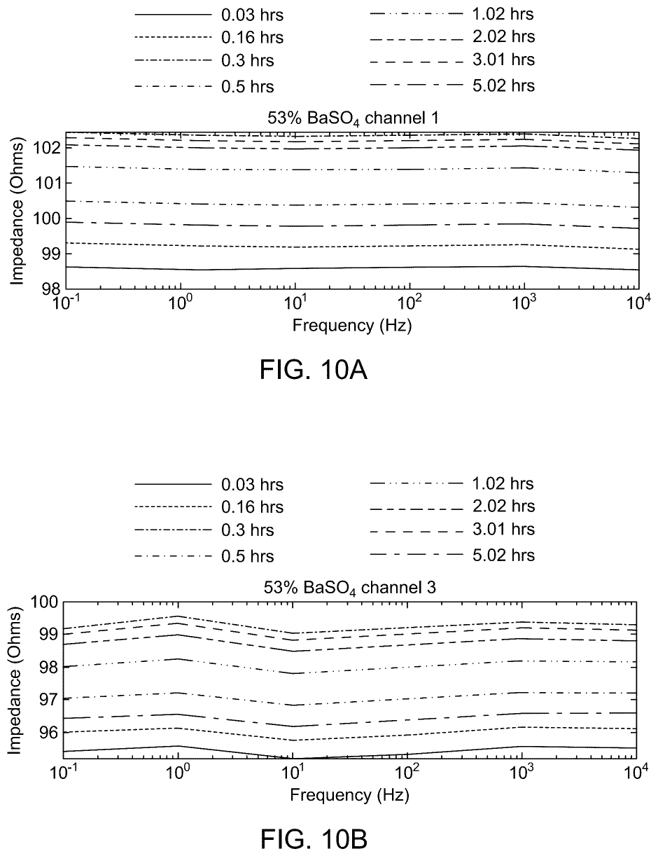

On the other hand, the spectrums of the real conductivity and the impedance (FIGS. 8A-8B and 10A-10B) exhibited the most significant alterations over the duration of the tests. These parameters are particularly sensitive to variations in concentration, which is closely linked to the intrinsic properties of fluids. Over time, a reduction in concentration was observed within the drilling fluid segments of channels 1 and 3, as a result of particle sedimentation. This trend is distinctly presented in FIGS. 8A-8B and 10A-10B, which illustrate the changes in the real conductivity and the impedance. Notably, there is an average reduction in the real conductivity by 3.9 μS/cm, coupled with an average increase in the impedance by 9.5 Ohms. These signal modifications are beneficial for monitoring the sedimentation of weighting materials in drilling fluids.

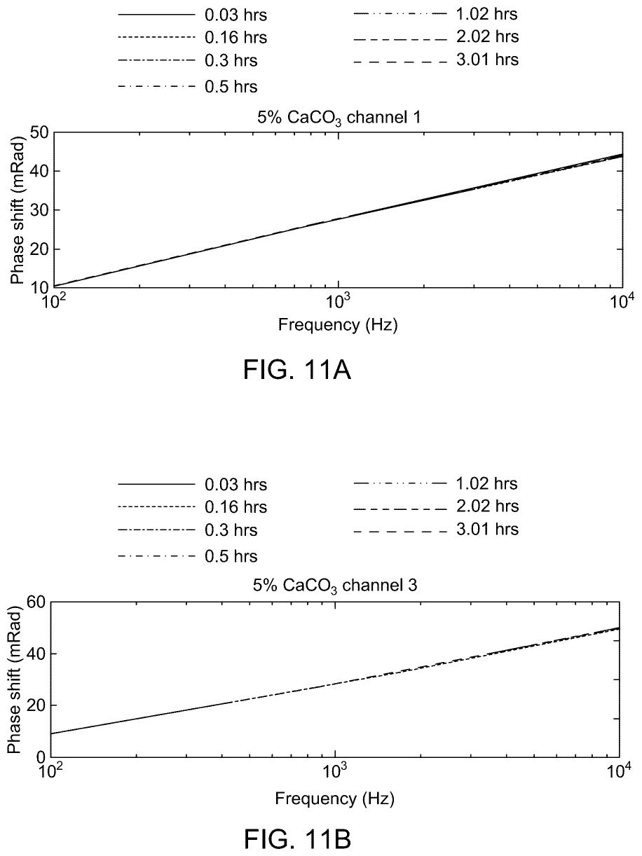

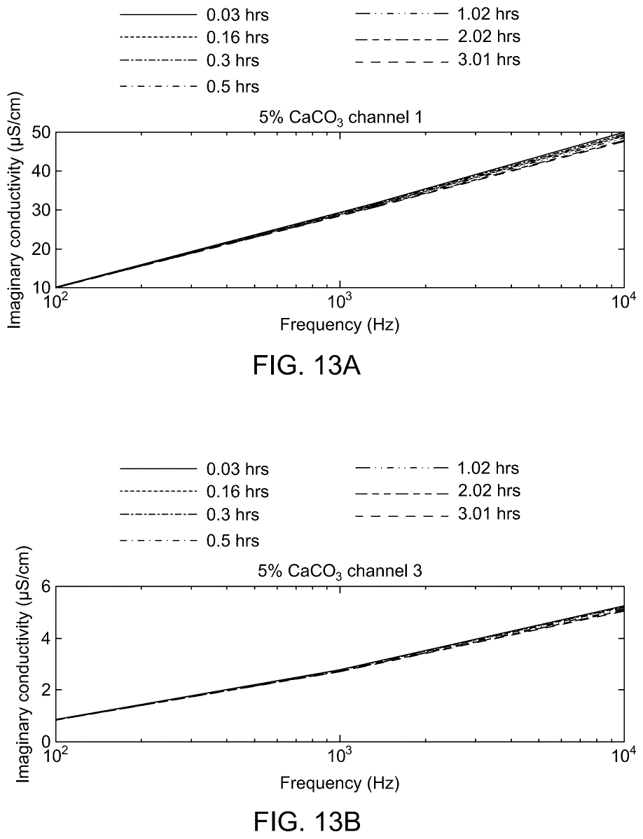

Further, given low electrical conductivity and limited capacity for charge storage of calcite, substantial fluctuations in the phase shift and the imaginary conductivity are not typically expected over time (as shown in FIGS. 11A-11B and 13A-13B). The variations that do occur in these parameters are proportional to the characteristics of the material being tested. Since the aggregate volume of solids present in the sample remains relatively constant and their conductive properties are modest, only slight shifts in both the phase shift and the imaginary conductivity are anticipated. These shifts are may be subtle in terms of their overall magnitude.

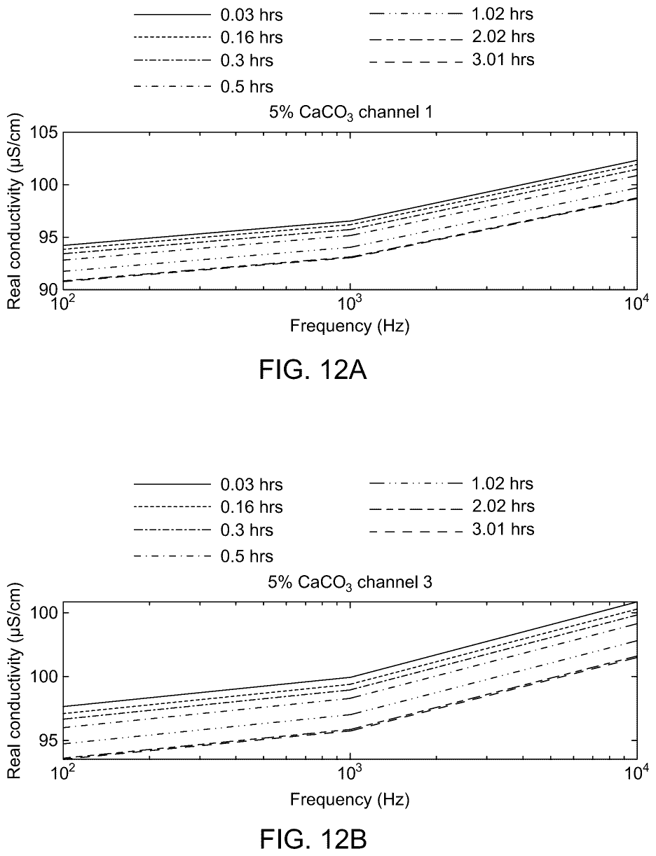

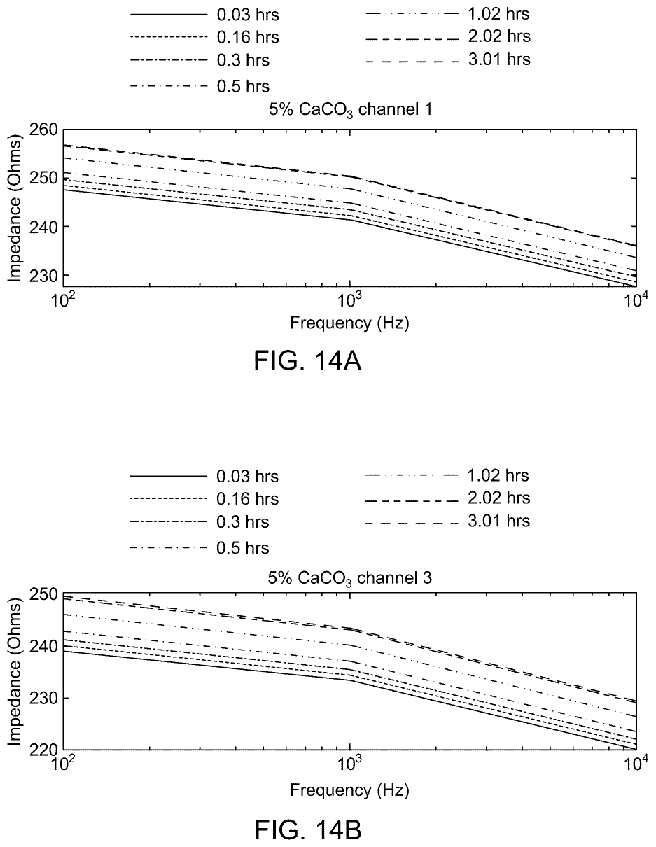

On the other hand, FIGS. 12A-12B and 14A-14B illustrate distinct patterns where the impedance rises and the real conductivity falls over time. This shift in behavior corresponds with the progression of time, as a reduction in particle concentration within the channels occurs due to sedimentation, prompting the observed changes in both signals. This trend is consistently evident across all frequency anchors within the time-lapse study. Similar to observations in barite drilling fluids, the real conductivity and the impedance in SIP measurements serve as key indicators for tracking the sag of weighting materials in drilling fluids.

The preceding data indicate that real conductivity and impedance offer the most definitive insights when monitoring the sag of weighting materials in water-based drilling fluids. Changes in these signals correlate with the alteration in particle concentration within the drilling fluid column over time. The real conductivity is indicative of the total electrical conductivity of the drilling fluids, encompassing both the liquid and solid constituents. With deionized water as the base fluid, the real conductivity predominantly arises from the solid additives of the drilling components. Therefore, a decrease in solid concentration due to settling is directly mirrored in the real conductivity signals. Conversely, the impedance gauges the resistance of the drilling fluid to electrical current. As such, the behavior of the impedance is inversely related to the real conductivity. A sharp rise in the real conductivity typically coincides with a corresponding decrease in the impedance.

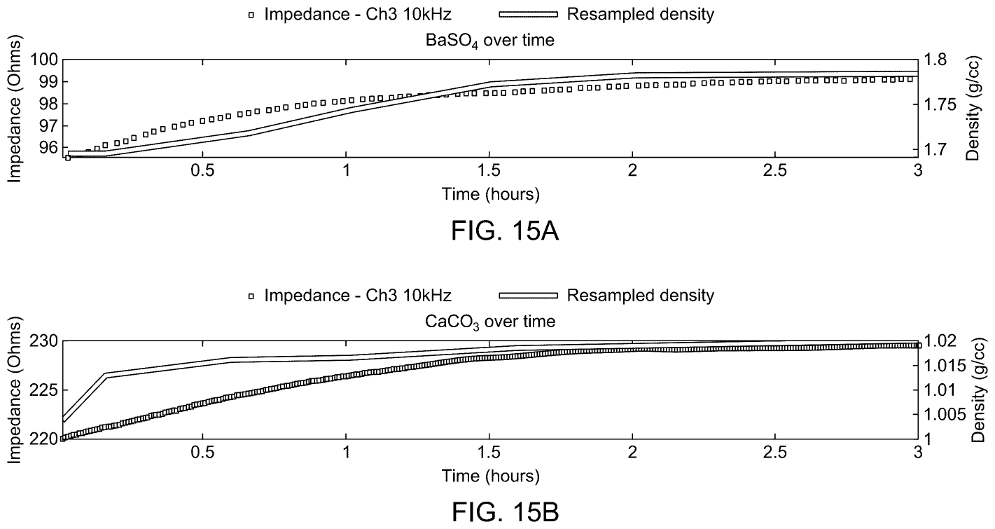

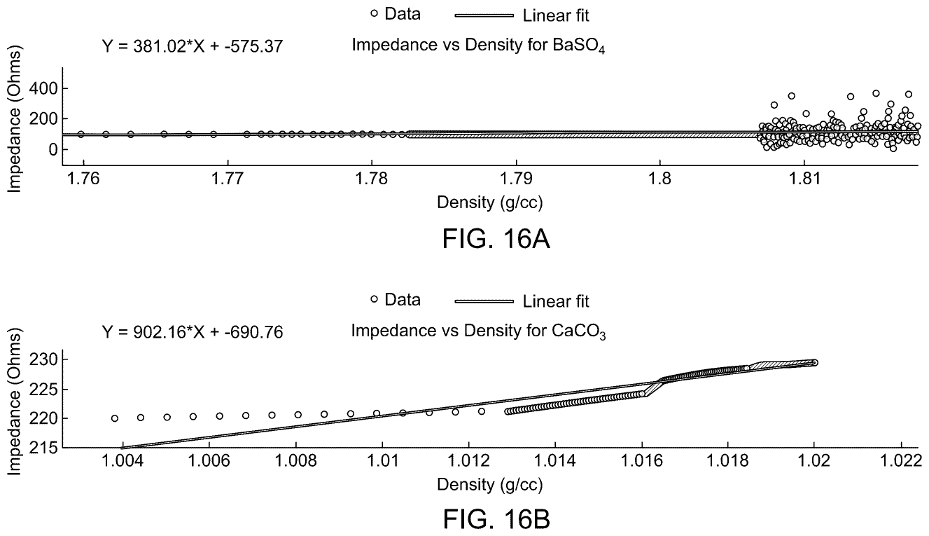

FIGS. 15A-15B and 16A-16B illustrate the impedance (in Ohms) and density (g/cc) of Channel 3, measured over time for CaCO₃ and BaSO₄. The graphs are intended to illustrate the relationship between impedance, density, and the settlement of particles in the column. The impedance of CaCO₃ increases sharply at first and then levels off, suggesting an initial rapid change in resistance of the channel to electrical current. This could be due to the quick settlement of CaCO₃ particles within the column, which might initially create a more compact layer, leading to a higher impedance. The density also rises and then plateaus, indicating that the mass per unit volume increases as the CaCO₃ particles settle. However, the density changes are relatively small, moving from just above 1.0 to just above 1.015 g/cc. Despite the significant change in impedance, this slight increase in density could mean that while the particles are settling and compacting, they do not contribute as much to the overall density, possibly due to the initial distribution of particles or the presence of other components in the channel. The same behavior was noticed for BaSO₄ with density increasing from about 1.7 to nearly 1.8 g/cc, which is a more noticeable change compared to the CaCO₃ graph.

The increases are not linear, they show a slight curvature, which indicates that the rate of change of both impedance and density could be accelerating or decelerating in the early stages. This could be due to a variety of factors such as rapid phase changes or filling process. For both substances, the relationship between impedance and density seems to be linked to the settlement of particles. As particles settle, they potentially create a more compact and uniform medium, which can increase both the electrical resistance (impedance) and the mass per unit volume (density). However, the rate of settlement and the final distribution of particles within the column seem to differ between CaCO₃ and BaSO₄, as indicated by the different patterns in impedance and density changes over time. Distinct discrepancies are observed between the signals of barite and calcite drilling fluids, stemming from inherently higher conductivity of barite. For CaCO₃, the impedance increases from about 222 Ohms to approximately 228 Ohms over the 3 hours; while for BaSO₄, the impedance increases from around 95 Ohms to just under 100 Ohms over 3 hours. The disparate composition of the two fluids also complicates direct signal comparison. In barite drilling fluids, the real conductivity exhibits more substantial changes than impedance, suggesting that barite particles are settling at the bottom of the testing channel. Since barite is more conductive than the other constituents, its removal has a pronounced impact. In contrast, the rise in impedance is less significant in barite than in calcite, as the remaining components maintain some conductivity even after barite particles have settled. For calcite drilling fluids, the increase in impedance is more marked, reflecting the scarcity of conductive materials once the calcite settles, particularly in the absence of stabilizing agents.

In general, overall, the real conductivity and the impedance measurements are highly sensitive techniques that detect even minor changes in the drilling fluid composition. Drilling engineers can closely monitor these parameters to identify sag in its nascent stages, allowing timely corrective actions. This early detection is crucial for preventing issues like stuck pipes or wellbore instability, which can lead to costly delays and accidents. When solid particles settle in the drilling fluid, the sag factor increases, indicating poor stability. As the sag factor increases, the concentration of solids in the fluid rises, affecting its electrical conductivity. An increase in the sag factor leads to a decrease in the real conductivity of the drilling fluid. This is because the settled solids create a barrier, hindering the flow of electric current through the fluid. Real conductivity measurements can, therefore, indirectly indicate the sag factor by detecting changes in ability of the drilling fluid to conduct electricity. Similarly, an increase in the sag factor results in a higher concentration of solids in the drilling fluid, leading to an increase in the impedance. Impedance, which includes both resistance and reactance, reflects the overall hindrance to the flow of electric current. As the sag factor increases, the settled solids create resistance and alter the dielectric properties of the drilling fluid, contributing to higher impedance values. Additionally, barite and calcite are common components of drilling fluids, each with unique properties. Real conductivity and impedance measurements enable the clear differentiation between these fluids. This differentiation is vital because the response of different fluids to sag might vary. It may be understood that other SIP parameters, including the phase shift and the imaginary conductivity, do not provide the same level of precision as the real conductivity and the impedance in this context. These parameters are more reflective of the charge storage capacity of solids. Given that the weighting materials in drilling fluids are non-metallic and do not have substantial charge storage or accumulation capabilities, the real conductivity and the impedance are the preferred metrics for monitoring sag as per embodiments of the present disclosure.

The present disclosure provides a method and system for monitoring barite and calcite sag, i.e., key weighting materials in drilling fluids, using Spectral Induced Polarization (SIP) over various time intervals. The SIP signals demonstrated a high degree of accuracy in tracking the sag of weighting materials over a two-hour period, after which the signals stabilized, indicating a slowdown in settlement. The most decisive indicators in this process were found to be the real conductivity and the impedance. These signals are needed due to the conductivity reduction in the system caused by particle migration. Compared to the phase shift and the imaginary conductivity, which are primarily linked to electric chargeability, the real conductivity and the impedance showed a more pronounced effect. The variations in the real conductivity and the impedance provide a framework for early detection of sag in the drilling fluid at ambient temperatures. For instance, the method 100 of the present disclosure could accurately detect slight particle settlements in both barite and calcite drilling fluids. In barite, a 1% and 2.6% settlement in the bottom and top segments of the drilling fluid, respectively, was indicated by an 11.5 μS/cm drop in the real conductivity and a 4.9 ohms increase in the impedance. Similarly, in calcite, a 2.8% and 4% settlement in the bottom and top segments, respectively, corresponded to a 3.9 μS/cm drop and a 9.5 ohms increase.

The results showing an increase in density and the impedance over time could be indicative of the settling or sagging of weighting materials in the drilling fluid. If such a trend is observed in real time, it could be used to predict early settling, allowing for proactive measures to prevent it. Settling can lead to variations in density throughout the wellbore, which can compromise well stability and even lead to stuck pipe situations. A stable drilling fluid density helps maintain hydrostatic pressure and wellbore stability. By using impedance measurements as a proxy for density changes, operators can ensure the wellbore is stable and reduce the risk of blowouts or collapse. Real-time monitoring of impedance and correlating it with density changes would allow for quick adjustments to the drilling fluid properties. This is crucial to maintaining the right mud weight for the specific drilling conditions and to adjust for changes in the formation being drilled.

The present disclosure provides early detection of issues such as settling and thus can prevent costly downtime and operational delays. The method and system of the present disclosure provide a means for maintaining proper control over drilling fluid properties and thereby reduce the risk of uncontrolled discharge of fluids into the environment. The method 100 of the present disclosure can be implemented downhole to provide a real-time tracking system, enabling the early identification of drilling fluid settlements. This advancement offers significant advantages in improving drilling operations and ensuring the stability of drilling fluids.

The method 100 is suitable for use in different well orientations, including vertical, inclined, and horizontal wells. The method can be employed at surface mud tanks and in downhole environments, demonstrating its adaptability in various drilling scenarios.

Numerous modifications and variations of the present disclosure are possible in light of the above teachings. It is therefore to be understood that within the scope of the appended claims, the invention may be practiced otherwise than as specifically described herein.

1. A method of detecting a sag value of a weighting material in a drilling fluid in a borehole, comprising:
injecting an alternating current into the drilling fluid in a frequency range of 0.1-10,000 hertz (Hz) over a time period of 1-6 hours;
measuring real-time data of a real conductivity and an impedance of the drilling fluid based on a spectral induced polarization (SIP) of the drilling fluid during the injecting;
calculating a first density and a second density of the drilling fluid at a first elevation and a second elevation, respectively, each based on a variation in the real conductivity and the impedance; and
calculating the sag value of the weighting material in the drilling fluid based on the first and second densities,
wherein the variation in the real conductivity and the impedance directly relates to changes in a concentration of particles of the weighting material in the drilling fluid.

2. The method of claim 1, wherein the method does not comprise extracting the drilling fluid from the borehole for the measuring.

3. The method of claim 1, wherein an amplitude of the alternating current is in a range of 1-10 volts (V) and wherein the injecting includes injecting the same alternating current into the drilling fluid at three different evenly spaced depths of the borehole.

4. The method of claim 1, wherein the frequency range is 1000-10,000 Hz, and wherein the time period is in a range of from 1-3 hours.

5. The method of claim 1, further comprising measuring a phase shift and an imaginary conductivity of the drilling fluid at different elevations of the borehole.

6. The method of claim 1, wherein the borehole has a depth of less than 1 km and wherein the injecting occurs concurrently in a top, a middle, and a bottom of the borehole.

7. The method of claim 1, further comprising calculating the concentration of the particles of the weighting material in the drilling fluid based on the real conductivity and the impedance.

8. The method of claim 1, wherein the drilling fluid comprises about 40-60 wt. % barite, based on a total weight of the drilling fluid.

9. The method of claim 8, wherein the drilling fluid further comprises 40-60 wt. % water, 0.1-1 wt. % of a polymer, and 0.1-1 wt. % of a starch, each based on the total weight of the drilling fluid.

10. The method of claim 8, wherein the first and second densities of the drilling fluid can be determined according to the equation d_{barite}=(Z+575.37)/381.02;
wherein d_{barite} denotes the first and second densities of the drilling fluid comprising barite, respectively; and
wherein Z denotes the first and second impedance of the drilling fluid, respectively.

11. The method of claim 1, wherein the drilling fluid comprises 1-10 wt. % calcite, based on a total weight of the drilling fluid.

12. The method of claim 11, wherein the drilling fluid further comprises 1-10 wt. % bentonite and 80-98 wt. % water, each based on a total weight of the drilling fluid.

13. The method of claim 11, wherein the first and second densities of the drilling fluid can be determined according to the equation d_{calcite}=(Z+690.76)/902.16;
wherein d_{calcite} denotes the first and second densities of the drilling fluid comprising calcite, respectively; and
wherein Z denotes the first and second impedance of the drilling fluid, respectively.

14. The method of claim 1, wherein the variation in the real conductivity is 1-20 μS/cm compared to an initial real conductivity.

15. The method of claim 1, wherein the variation in the impedance is 1-20 ohms compared to an initial impedance.

16. The method of claim 1, wherein the drilling fluid has a salinity of 1 to 1000 ppt.

17. The method of claim 1, wherein the drilling fluid is in a reservoir and the reservoir has a temperature of 100-300° C.

18. The method of claim 1, wherein the drilling fluid is in a subterranean borehole having a depth of at least 500 m during the injecting and the measuring.

19. The method of claim 1, wherein the drilling fluid is in a horizontal well during the injecting and the measuring.

20. The method of claim 1, wherein the measuring is downhole in a well during the injecting and the measuring.
